(19) United States
(12) Patent Application Publication
(10) Pub. No.: US 2012/0288879 A1
(43) Pub. Date: Nov. 15, 2012

(54) METHODS FOR DEVELOPING AND ASSESSING THERAPEUTIC AGENTS

(75) Inventor: Soner Altiok, Tampa, FL (US)

(73) Assignee: Altiok, Inc., Tampa, FL (US)

(21) Appl. No.: 13/471,075

(22) Filed: May 14, 2012

Related U.S. Application Data
(63) Continuation of application No. 12/308,005, filed on Jul. 16, 2009.
(60) Provisional application No. 60/811,038, filed on Jun. 5, 2006.

(51) Int. Cl. G01N 33/566; G01N 33/574

(52) U.S. Cl. 435/7.92; 435/7.1

(57) ABSTRACT

Assays are provided that can effectively assess tumor response to one or more therapeutic agents. Preferred assays of the invention include assessment of posttranslation modification and expression of target proteins.

### CROSS-REFERENCE TO RELATED APPLICATION

This application which is a continuation of application Ser. No. 12/308,005 which was filed on Jul. 16, 2009 is a continuation of PCT/US2007/01310 which was filed on Jun. 4, 2007 which claims the benefit of U.S. Provisional Application No. 60/811,036 which was filed on Jun. 4, 2006, the entire disclosures of which are hereby incorporated in their entirety.

### BACKGROUND OF THE INVENTION

1. Field of the Invention

The present invention includes assays that can effectively assess tumor response to one or more therapeutic agents. Preferred assays of the invention include assessment of posttranslation modification and expression of target proteins.

2. Background

Progress in understanding the molecular biology of cancer has provided an abundant source of potential therapeutic targets. This has, in turn, fostered the development of an unprecedented number of novel drugs available for clinical testing. The exquisite selectivity of these agents renders them capable of specifically targeting critical nodes in the cellular signaling pathways now understood to be dysfunctional in cancer cells. Nevertheless, it is becoming increasingly evident that traditional drug development paradigms may not be ideally suited to realize the full clinical potential of these new agents. One logical organizing principle is that targeted therapeutics will be effective against tumors in which the target is biologically important and is adequately blocked by the drug (1-3).

The development of targeted anticancer agents requires integration of new pharmacodynamic and surrogate end points into clinical trials to demonstrate that the targeted drugs lead to inhibition of the biological targets at doses that are well tolerated, and that the consequences of target inhibition can be identified and measured using validated surrogates for clinical benefit.

The development of imatinib mesylate for chronic myelogenous leukemia and gastrointestinal stromal tumors offers one example of a situation where conventional taxonomic schemes corresponded to a critical and, in this case, effectively treatable molecular target in a preponderance of cases (4, 5). Somewhat in contrast, more recent experience has been characterized by the striking differential efficacy of EGFR-targeted agents among subgroups of solid tumor patients with distinct molecular profiles (6-12). This experience has begun to offer the insight that tools for rational patient selection may provide a key means to define these biologically discrete subpopulations of patients and better realize the potential of these agents for larger numbers of patients (13-15). One principal limitation in this area is the lack of sophisticated preclinical models permitting the development of tissue acquisition protocols and candidate biomarkers predictive of drug actions. Surrogate tissues, such as peripheral blood mononuclear cells, skin, and hair follicles have been used to monitor therapy effect in immunohistochemical or kinase studies. However, frequently, preparing the surrogate tissues can be time consuming and/or technically challenging that may involve relatively invasive core biopsy sampling with discomfort to the patient. Therefore sampling can be obtained in only a limited proportion of patients and at a small number of time points to monitor therapy effect.

Despite substantial progress made in recent years, there are currently no clinically validated tests to predict the efficacy of a given agent for an individual patient. While there is consensus supporting the need to develop and integrate the evaluation of predictive biomarkers in clinical trials, the practical application of such an approach is still lagging behind (16). Three main issues define the obstacles in the way of realizing this conceptual goal. As a start, robust and well-validated assays that faithfully predict treatment outcomes are needed. Next, such assays must be applicable to readily available clinical materials. Finally, there is a need to develop practical, minimally morbid means of collecting reliable yields of tumor material in a serial manner for correlation of biomarker readout with clinical response over time.

### SUMMARY OF THE INVENTION

Methods are provided that can effectively assess response to one or more therapeutic agents such as those that may target epidermal growth factor receptor (EGFR), MEK1/2, MAPK/ERK1/2, AKT/PKB and/or m-TOR in cancer cells. Effectiveness of the assays of the invention have been demonstrated in human subjects.

Accordingly, in one aspect the invention provides, a method for assessing the therapeutic potential of one or more chemotherapeutic or metabolic agents, the method comprising obtaining a subject sample, treating the subject sample with the one or more candidate therapeutic agents, and then determining the expression or activation of signaling or metabolic proteins in the subject sample, wherein an alteration in the level of expression or activation of the proteins in the subject sample relative to the level of expression or activation in a reference sample indicates the therapeutic potential of one or more chemotherapeutic or metabolic agents.

In one embodiment, the method is carried out prior to or during a therapeutic treatment regime. In another embodiment, the treatment regimen is for a neoplasia or a metabolic disease or disorder.

In another aspect, the invention features a method of monitoring a subject diagnosed as having a neoplasia or a metabolic disease or disorder, the method comprising determining the expression or activation of signaling or metabolic proteins in a subject sample, wherein an alteration in the level of expression or activation of the proteins in the subject sample relative to the level of expression or activation in a reference sample indicates the severity of the neoplasia or the metabolic disease or disorder.

In one embodiment, the subject sample is taken before, and at one or more time points after the start of a therapeutic treatment regimen.

In another embodiment, the subject sample is a biological sample.

In a further embodiment, the subject sample is taken from a subject suffering from a neoplasia. In a related embodiment, the neoplasia is selected from the group consisting of: bladder, breast, colon, endometrial, kidney, renal, rectal, leukemia, lung, melanoma, pancreatic, prostate, skin, thyroid, and ovarian.

In one embodiment, the subject sample comprises tumor cells.

In another embodiment, the subject sample is taken from a subject suffering from diabetes or obesity.

In another embodiment, the subject sample is adipose cells.

In one embodiment, the method is performed ex vivo. In a related embodiment, the method is performed in vivo.

In one embodiment, the tumor cells are obtained by fine needle aspiration biopsy (FNAB).

In one embodiment, the tumor cells are obtained by a biopsy. In a related embodiment, the biopsy is an endoscopic, surgical or fat pad biopsy.

In one embodiment of the above aspects, the alteration is an increase, and the increase indicates an increased severity of the neoplasia or the metabolic disease or disorder.

In another embodiment, the reference is a subject sample that is not being treated for a neoplasia or a metabolic disorder.

In a related embodiment, the reference is a subject sample obtained at an earlier time point.

In a further embodiment, the method is used to diagnose a subject as having a neoplasia or a metabolic disorder.

In another embodiment, the method is used to determine the treatment regimen for a subject having a neoplasia or a metabolic disorder.

In another embodiment, the method is used to monitor the condition of a subject being treated for a neoplasia or a metabolic disorder.

In one embodiment, the method is used to determine the prognosis of a subject having a neoplasia or a metabolic disorder. In a related embodiment, a poor prognosis determines an aggressive treatment regimen for the subject.

In another embodiment, the subject sample or subject is treated with one or more chemotherapeutic agents or one or more metabolic agents.

In one embodiment, the subject or subject sample is treated with one or more ERK, MEK 1/2, MAPK, AKT/PKB or mTOR inhibitory compounds.

In another embodiment, the subject or subject sample is treated with one or more HDAC inhibitory compounds.

In a further embodiment, determining the activation of signaling or metabolic proteins in the subject sample comprises determining the phosphorylation status if one or more enzymes or proteins in the subject sample.

In another embodiment, the expression and phosphorylation status of one or more EGFR signaling proteins is assessed.

In a further embodiment, the expression and phosphorylation status of one or more MEK/ERK signaling proteins is assessed.

In a related embodiment, the expression and phosphorylation status of one or more MAPK signaling proteins is assessed.

In another embodiment, the expression and phosphorylation status of one or more JNK signaling proteins is assessed.

In another embodiment, protein acetylation is assessed.

In one embodiment, activity of one or more histone deactylase (HDAC) enzymes are assessed.

In another embodiment, expression levels of one or more proteins are assessed.

In one embodiment, the one or more chemotherapeutic or metabolic agents is selected from the group consisting of: abarelix; aldesleukin; Aldesleukin; Alemtuzumab; alitretinoin; allopurinol; altretamine; amifostine; anastrozole; arsenic trioxide; asparaginase; azacitidine; BCG Live; bevacuzimab; bexarotene; bexarotene; bleomycin; bortezomib; busulfan; calusterone; capecitabine; carboplatin; carmustine; celecoxib; cetuximab; chlorambucil; cisplatin; cladribine; clofarabine; cyclophosphamide; cytarabine; dacarbazine; Darbepoetin alfa; daunorubicin; decitabine; Denileukin diftitox; dexrazoxane; docetaxel; Dromostanolone; doxorubicin; Elliott's B Solution; epirubicin; Epoetin alfa; erlotinib; estramustine; etoposide phosphate; etoposide; exemestane; Filgrastim; floxuridine; fludarabine; fluorouracil, 5-FU; fulvestrant; gefitinib; gemcitabine; gemtuzumab ozogamicin; goserelin; histrelin; hydroxyurea; Ibritumomab Tiuxetan; idarubicin; ifosfamide; imatinib; interferoninterferon; irinotecan; lenalidomide; letrozole; leucovorin; Leuprolide Acetate; levamisole; lomustine; meclorethamine; megestrol; melphalan, L-PAM; mercaptopurine, 6-MP; mesna; methotrexate; methoxsalen; mitomycin C; mitotane; mitoxantrone; nandrolone phenpropionate; nelarabine; Nofetumomab; Oprelvekin; oxaliplatin; paclitaxel; palifermin; pamidronate; pegademase; pegaspargase; Pegfilgrastim; pemetrexed; pentostatin; pipobroman; plicamycin, mithramycin; porfimer; procarbazine; quinacrine; Rasburicase; Rituximab; sargramostim; sorafenib; streptozocin; sunitinib; talc; tamoxifen; temozolomide; teniposide, VM-26; testolactone; thioguanine, 6-TG; thiotepa; topotecan; toremifene; Tositumomab; Tositumomab/I-131 tositumomab; Trastuzumab; tretinoin, ATRA; Uracil Mustard; valrubicin; vinblastine; zoledronate; and zoledronic acid.

In another related embodiment, the one or more chemotherapeutic or metabolic agents is selected from the group consisting of: Panitumumab; Erbitux; Temsiroliumus; Avastin; Tykerb; Herceptin; and Sutent.

In another aspect, the invention features a method of identifying a compound that inhibits a neoplasia or a metabolic disease or disorder, the method comprising determining the expression or activation of signaling or metabolic proteins in a cell, contacting the cell with a candidate compound, and then comparing the level of expression or activation of signaling or metabolic proteins in the cell contacted by the candidate compound with the level of expression in a control cell not contacted by the candidate compound wherein an alteration in the level of expression or activation of the proteins in the subject sample relative to the level of expression or activation in a reference sample not contacted with compound identifies a compound that inhibits a neoplasia or a metabolic disease or disorder.

In one embodiment, the alteration is an increase.

In one embodiment, determining the activation of signaling or metabolic proteins in the subject sample comprises determining the phosphorylation status if one or more enzymes or proteins in the subject sample.

In another embodiment, the expression and phosphorylation status of one or more EGFR signaling proteins is assessed.

In another embodiment, the expression and phosphorylation status of one or more MEK/ERK signaling proteins is assessed.

In another embodiment, the expression and phosphorylation status of one or more MAPK signaling proteins is assessed.

In another embodiment, the expression and phosphorylation status of one or more JNK signaling proteins is assessed.

In another embodiment, protein acetylation is assessed.

In one embodiment, activity of one or more histone deactylase (HDAC) enzymes are assessed.

In another embodiment, expression levels of one or more proteins are assessed.

In one embodiment, the cell is in vitro. In one embodiment, the cell is in vivo.

In another embodiment, the cell is a human cell.

In another embodiment, the cell is a neoplastic cell or an adipose cell.

Other aspects of the invention are discussed below.

### BRIEF DESCRIPTION OF THE DRAWINGS

FIG. 1 shows RTG of individual patient tumors in xenograft mice treated with erlotinib (black bar) or temsirolimus (dashed bar). RTG was determined for each individual tumor, as described in the method section.

FIG. 2 shows representative tumor growth and ex vivo assay data from a temsirolimus susceptible (A198) and resistant (A194) pancreatic cancer tumor. Exposure to 1 μM of temsirolimus ex vivo inhibited phosphorylation of S6-RP in the tumor cells obtained from the susceptible tumor (A198), but not from the resistant tumor (A194).

FIG. 3 shows representative tumor growth and ex vivo assay data from an erlotinib susceptible (A198) and a resistant (A265) pancreatic cancer tumor. Treatment of tumors cells with 5 μM of erlotinib ex vivo inhibited ERK1/2 activation in the tumor cells obtained from the susceptible tumor (A198), but not from the resistant tumor (A265).

FIGS. 4 (A and B) shows: A) Ex vivo (upper panel) and in vivo (lower panel) studies with temsirolimus in six different xenograft mice bearing primary human pancreatic carcinoma tumors. B) Representative INC staining of phospho-p70S6k (P-pS6K) and total-p70S6K (T-pS6K) in two representative tumors, A198 and A194, treated with vehicle or temsirolimus.

FIGS. 5 (A and B) shows: A) Ex vivo (upper panel) and in vivo (lower panel) studies with erlotinib in six different xenograft models of human pancreatic cancer. B) Representative IHC staining of phospho- and total-ERK1/2 in two representative tumors, A198 and A265, treated with vehicle or erlotinib.

FIG. 6 shows: Tumor FNAB obtained from cancer patients during routine diagnostic procedures provide tumor samples with adequate cellularity to perform ex vivo drug sensitivity assays. Tumor FNAB samples were collected from three pancreatic cancer patients (AD/DQ). Tumor cells were treated ex vivo with vehicle (control), temsirolimus or erlotinib for 6 hours. Whole-cell extracts were prepared and total expression (T-) and phosphorylation (P-) levels of ERK1/2 or S6-RP were analyzed on Western blot.

FIG. 7 shows: Air-dried and Diff-Quick (AD/DQ) stained cytologic slides of T-47D cell lines allow detection of expression levels and phosphorylation status of EGFR and ERK1/2 proteins. T-47D cells were serum starved and either untreated (−) or treated (+) with EGF (100 ng/ml) for 15 minutes. Whole cell lysates directly from cultured cells (control) or from AD/DQ cytologic slides. Protein expression and phosphorylation levels were determined by Western blot with antibodies prepared against phosphor-EGFR (Cell signaling, #2334), total-EGFR (Cell Signaling, #2232), phosphor-ERK1/2 (Cell signaling, #9101) and total ERK1/2 (Cell signaling, #9102).

FIGS. 8 (A and B) shows: Air-dried cytologic samples high quality proteins to analyze ERK1/2 activity by ELISA in quantitative manner. A) Phospho- and total ERK1/2 ELISA can detect treatment-mediated changes in the phosphorylation of ERK1/2 in AD/DQ T-47 cytologic smears (raw data (left), normalized (right) B) shows corroboration of ELISA results by Western blot analysis.

FIG. 9 shows: FNAB samples of HUCCT-1 tumors provide highly enriched tumor ell populations.

FIG. 10 shows: Ex vivo treatment of human breast cancer cells allows assessment of tumor response to targeted inhibitors. Tumor cells were treated with DMSO (control), or inhibitors of PI3K/AKT, MEK/ERK1/2 or EGFR ex vivo. Total levels and phosphorylation states of target proteins were analyzed by Western blot.

FIG. 11 shows: In vivo tumor response to targeted therapies were assed ex vivo in neoplastic cells obtained by tumor FNAB. A: Tumor cells were colleted by FNAB before the initiation of therapy and treated ex vivo with ZD1839 or CI-799 for 6 hours. Cell lysates were prepared and analyzed for phosphor- and total ERK1/2 or S6 ribosomal protein (S6 RBP) on Western Blot. FNAB samples were obtained before (day 0) and during (day 7) the therapy from the same animals tested for ex vivo sensitivity, as described in the upper panel. Protein samples were prepared from AD/DQ slides and phosphorylation status as well as total levels of ERK1/2 and S6 ribosomal protein (S6 RBP) were determined on Western blot. The results were correlated with therapy mediated changes in tumor volume.

FIG. 12 shows: Air-dried and Diff-Quik (AD/DQ)-stained cytologic slides of T47D cell lines allow detection of expression levels and phosphorylation status of EGFR and ERK1/2 proteins. T47D were serum starved and either untreated (−) or treated (+) with EGF (100 ng/ml) for 15 min. Whole cell lysates were prepared directly from cultured cells (control) or from AD/DO-stained cytologic slides. Protein expression and phosphorylation levels were determined by Western blot with antibodies prepared against phospho-EGFR, total-EGFR, phospho-ERK1/2, and total ERK1/2.

FIGS. 13 (A and B) shows: Air-dried, Diff-Ouik-stained cytologic samples yield high quality proteins to analyze ERK1/2 activity by ELISA in a quantitative manner. A) Phospho- and total ERK1/2 ELISA can detect treatment-mediated changes in the phosphorylation of ERK1/2 in AD/DO-stained T47D cytologic smears, (raw data, upper graph; normalized data, lower graph) B) Corroboration of ELISA results by Western blot analysis. The experiment was performed three times with similar results.

FIGS. 14 (A and B) shows: FNAB samples of HUCCT-1 tumors provide highly enriched tumor cell populations. A) FNAB samples (AD/DO), B) Resection specimen (hematoxylin and eosin stain).

FIG. 15 shows antiproliferative effects of gefitinib and CI-1040 against HuCCT-1 tumors in nude mice. Animals were treated with gefitinib and CI-1040 alone and in combination for 2 consecutive weeks. Data represent tumor volume and SE.

FIG. 16 (A-C) shows: A) Tumor FNAB samples detect therapy-mediated changes in the phosphorylation of EGFR and ERKI/2 in vivo. Cell lysates were prepared from AD/DQ FNAB slides and protein phosphorylation (P-) and total expression (T-) levels of EGFR and ERK1/2 proteins were determined on Western blot analysis by using antibodies described in the legend of FIG. 1. Results were correlated with therapy-induced changes in tumor size. B) Early FNAB sampling can predict tumor response in vivo. Protein extracts were prepared from FNAB slides and phosphorylation and expression levels of ERK1/2 proteins were determined on Western blot analysis. C) Detection and quantification of total and phospho-ERK1/2 in tumor FNAB samples. Cell extracts were prepared from FNAB smears in 0.1% SDS, boiled, and analyzed in the ERK1/2 [pTpY185/187J phosphoELISA and ERK1/2 (Total) ELISA, (raw data, upper graph; normalized data, lower graph). These experiments were repeated at least three times, and one representative result is shown.

FIGS. 17 (A through H) show results of human tumor assessments with Iressa (gefitinib) in accordance with assays of the invention.

FIG. 18 shows chemotherapeutic assessment by evaluation of H3 acetylation and ERK inhibition in a cancer patient.

FIGS. 19 (A and B). Panel A shows a schematic representation of a fat pad biopsy. Panel B shows air-dried/Diff-Quick-stained smear sample obtained by fat pad fine needle aspiration biopsy (FNAB).

FIG. 20 shows Western blot analysis showing phosphorylation of signaling proteins in fat pad biopsy samples.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “alteration” is meant an increase or a decrease.

By “neoplasia” is meant any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “protein” is meant any chain of amino acids, or analogs thereof, regardless of length or post-translational modification.

By “reference” is meant a standard or control condition.

By “subject” is meant a vertebrate, preferably a mammal, more preferably a human. Mammals include, but are not limited to, murines, simians, humans, farm animals, sport animals, and pets.

By “prevent,” “preventing,” “prevention,” “prophylactic treatment” and the like are meant to refer to reducing the probability of developing a disorder or condition in a subject, who does not have, but is at risk of or susceptible to developing a disorder or condition.

By “increased” means a positive alteration. Exemplary increases include 2-fold, 5-fold, 10-fold, 20-fold, 40-fold, or even 100-fold.

By “aggressive treatment regimen” is intended to mean reducing or ameliorating a disorder and/or symptoms associated therewith a method of treatment (e.g. combination of chemotherapeutic agents) more intensive or comprehensive than usual, for instance in dosage or extent. It will be appreciated that, although not precluded, aggressively treating a disorder or condition does not require that the disorder, condition or symptoms associated therewith be completely eliminated.

By “metabolic disorder” is intended to include any disorder affecting a cell's metabolism. Exemplary metabolic disorders include obesity, and diabetes, including type I and type II diabetes.

By “epidermal growth factor receptor (EGFR) inhibitor” or “EGFR inhibitory compound” is intended to refer to compounds that decrease or otherwise interfere with the activity of the EGFR signaling or the EGFR receptor under normal or disease conditions.

By “mitogen activated kinase (MAPK) inhibitor” or “MAPK inhibitory compound” is intended to refer to compounds that decrease or otherwise interfere with the activity of MAPK signaling under normal or disease conditions.

By “extracellular signal-regulated kinase (ERK) inhibitor” or “ERK inhibitory compound” is intended to refer to compounds that decrease or otherwise interfere with the activity of ERK signaling under normal or disease conditions.

By “Jun N-terminal kinase (JNK) inhibitor” or “JNK inhibitory compound” is intended to refer to compounds that decrease or otherwise interfere with the activity of JNK signaling under normal or disease conditions.

By “Akt or protein kinase B (PKB)” or “AKT/PKB inhibitory compound” is intended to refer to compounds that decrease or otherwise interfere with the activity of AKT/PKB signaling under normal or disease conditions.

By “mammalian target of rapamycin (mTOR)” or “mTOR inhibitory compound” is intended to refer to compounds that decrease or otherwise interfere with the activity of mTOR signaling under normal or disease conditions.

By “tumor” is intended to include an abnormal mass or growth of cells or tissue. A tumor can be benign or malignant.

By “histone deacetylase inhibitors (HDAC inhibitors)” is meant a class of compounds that are able to modulate transcriptional activity. HDAC inhibitors can, in certain examples, block angiogenesis and cell cycling, and promote apoptosis and differentiation. HDAC inhibitors may modulate chromatin plasticity, facilitating protein:DNA interactions and thus transcriptional control.

By “therapeutic potential” is meant the ability of an agent to reducing or ameliorating a disorder and/or symptoms associated therewith. It will be appreciated that, although not precluded, the therapeutic potential does not require that the disorder, condition or symptoms associated therewith be completely eliminated.

By “therapeutic treatment regime” is meant to include the agents or combination of agents used to treat a subject. A therapeutic treatment regime may comprise 1, 2, 3, 4 or more agents at any given time.

It has now been found that tumor cells such as obtained by fine needle aspiration, scraping of resection specimens or from endoscopic biopsies, or other means are viable and can be used to predict response to targeted therapeutics and to conventional chemotherapeutics prior to initiation of therapy. In preferred methods, this can be done by treating cells ex vivo for short period of time with drugs and analyzing at the molecular levels drug mediated changes in the posttranslational modification (phosphorylataion, acetylation etc) and expression of target proteins. In additional preferred methods, assessment can be made in vivo, and combinations of ex vivo and in vivo assessments also may be employed.

It also has now been found that cellular proteins isolated from tumor cells and their stained or unstained cytologic smears obtained by e.g. fine needle aspiration, scraping of resection specimens or from endoscopic biopsies, or other means can provide adequate samples that are employed to analyze therapy mediated changes in the quality (phosphorylation, acetylation etc) and quantity of cellular proteins by proteomic assays such as Western blot, ELISA, mass spectrometry and quantitative enzymatic fluorescent assays, or other means.

Fat pad biopsy is a relatively noninvasive, economical, and fast procedure and commonly used to analyze amyloid deposition by Congo Red staining in routine pathology practice. However, phospho-proteomic analysis of cellular signaling in fat pad biopsies has never been explored before. It has now been found that fat pad biopsy materials yield high quality protein to assess the phosphorylation status of key signaling pathway elements.

Included in the invention are methods that can be used to assess the therapeutic potential of one or more chemotherapeutic or metabolic agents. These methods involve obtaining a subject sample, treating the subject sample with the one or more candidate therapeutic agents; and then determining the expression or activation of signaling or metabolic proteins in the subject sample, where an alteration in the level of expression or activation of the proteins in the subject sample relative to the level of expression or activation in a reference sample indicates the therapeutic potential of one or more chemotherapeutic or metabolic agents.

Also included in the invention are methods for monitoring a subject diagnosed as having a neoplasia or a metabolic disease or disorder. The methods comprise, for example, determining the expression or activation of signaling or metabolic proteins in a subject sample, where an alteration in the level of expression or activation of the proteins in the subject sample relative to the level of expression or activation in a reference sample indicates the severity of the neoplasia or the metabolic disease or disorder.

The invention as described herein is also useful for identifying a compound that inhibits a neoplasia or a metabolic disease or disorder. The method comprises determining the expression or activation of signaling or metabolic proteins in a cell, and contacting the cell with a candidate compound, and comparing the level of expression or activation of signaling or metabolic proteins in the cell contacted by the candidate compound with the level of expression in a control cell not contacted by the candidate compound, where an alteration in the level of expression or activation of the proteins in the subject sample relative to the level of expression or activation in a reference sample not contacted with compound identifies a compound that inhibits a neoplasia or a metabolic disease or disorder.

The method of the invention may be carried out before a subject undergoes a treatment regime. In this way, the method can be used to determine the efficacy of the treatment regime. In certain preferred examples, the method is carried out prior to or during a therapeutic treatment regime. Further, serial samples may be taken, that is serial subject samples from before, and at different time points during the treatment regime, for example, and then used in the methods of the invention to assess the efficacy of the treatment.

It has been demonstrated that methods and assay of the invention are effective to assess susceptibility of human tumors to candidate chemotherapies.

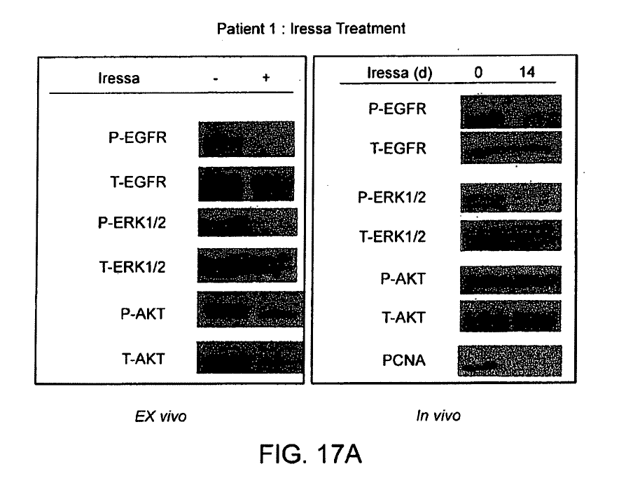

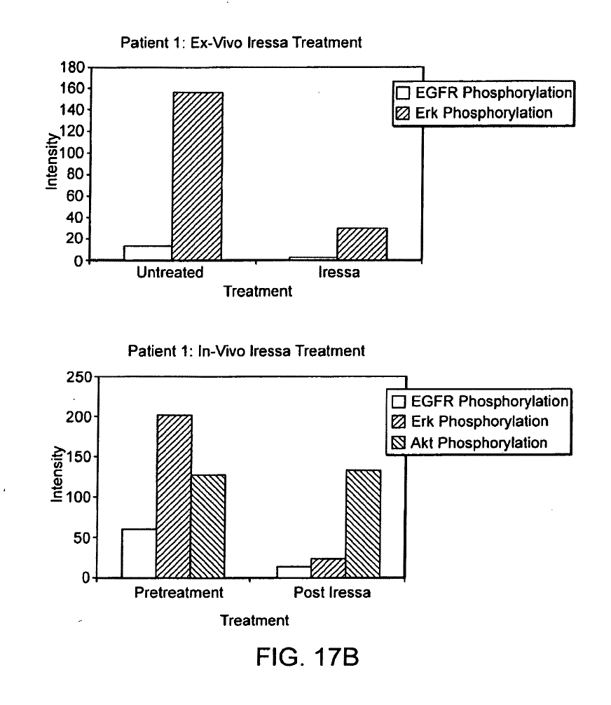

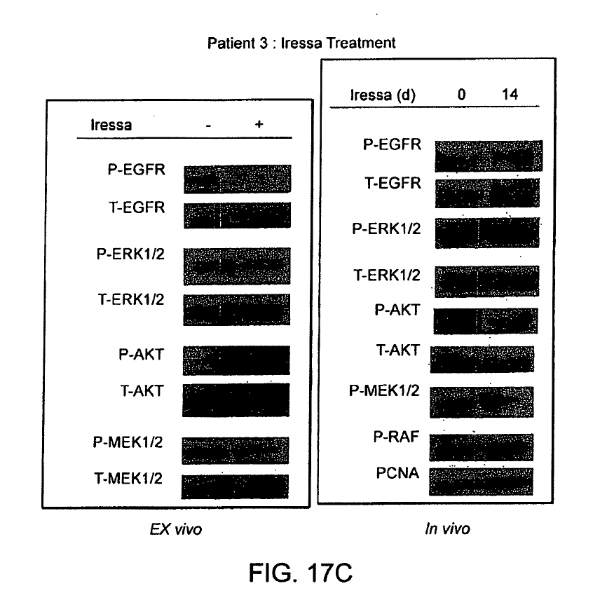

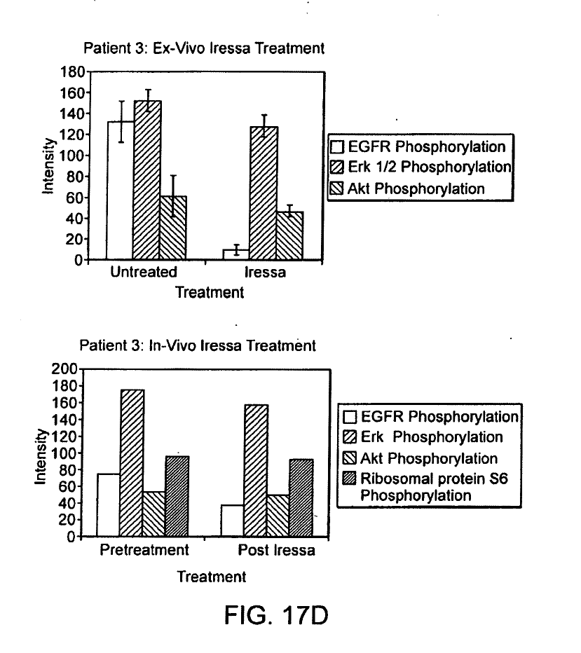

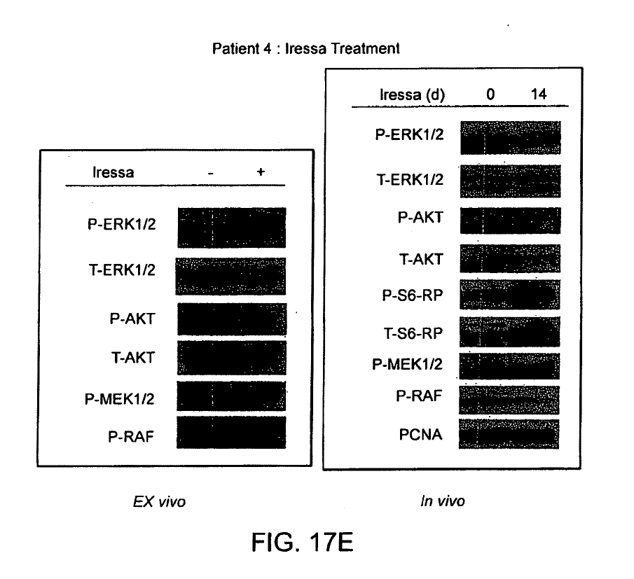

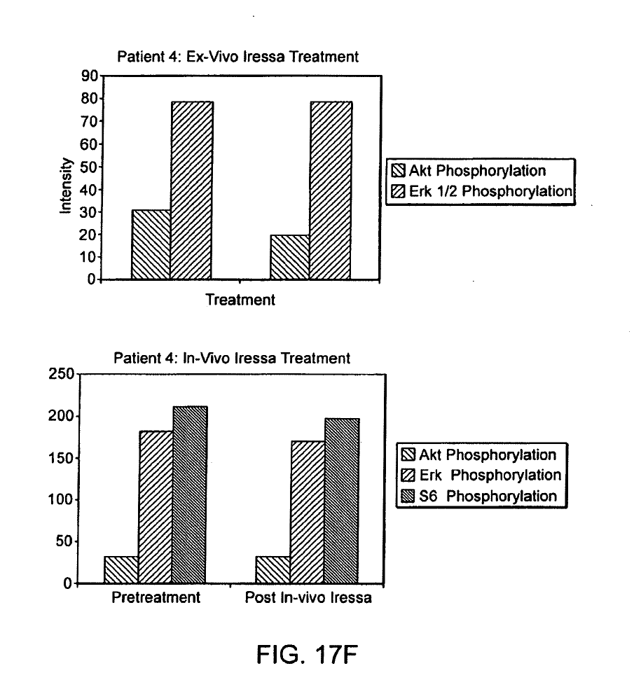

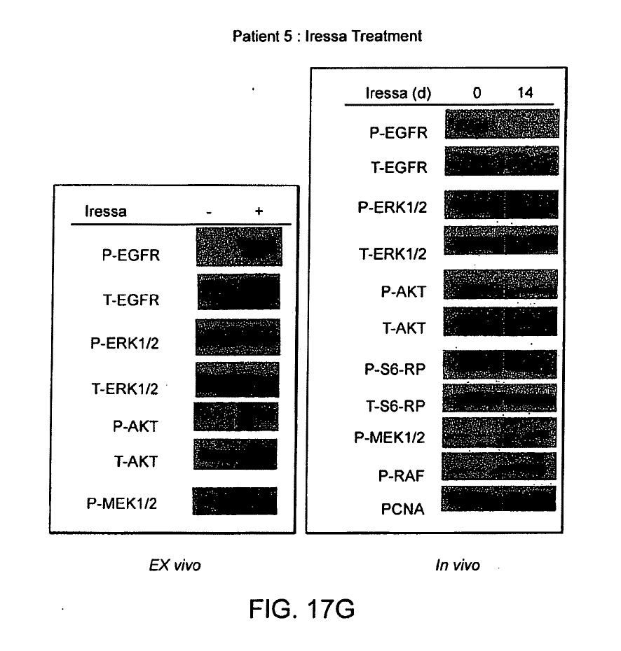

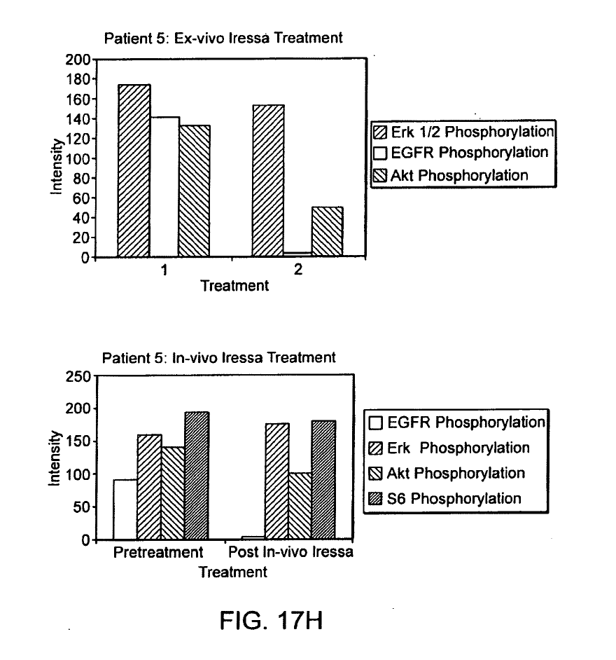

In particular, in one study, results of ex vivo and in vivo assays as disclosed herein from multiple esophageal cancer patients (human) showed high correlation between pretreatment prediction ex vivo and post-treatment monitoring in vivo. See also Example 12 which follows and related FIGS. 17 A though H.

In the methods and assay of the invention, tumor samples may be treated with one or more of a variety of candidate therapeutic agents or protocols to assess therapeutic potential of such compounds and protocols including e.g. one or more ERK blocker compounds; one or more HDAC inhibitor compounds, and the like. Preferred candidate therapeutic agents also may include agents that can target epidermal growth factor receptor, MEK1/2, MAPK/ERK1/2, AKT/PKB and/or m-TOR in cancer cells.

While a variety of candidate drug-mediated changes can be assessed in methods a assay of the invention, in preferred systems, posttranslational modification (phosphorylataion, acetylation etc) and expression of target proteins may be assessed. For instance, modulation of the ERK pathway may be is assessed. Phosphorylation status of one or more enzymes also may be assessed, such as phosphorylation status of one EGFR signaling proteins. Protein acetylation also may be assessed.

Thus, for instance, the degree of inhibition in the phosphorylation of target proteins in response to treatment of tumor cells with candidate therapy has correlated well with changes in tumor volume and decrease in PCNA expression in vivo, i.e. xenograft animals sensitive to therapy have shown the highest average inhibition of target protein phosphorylation, whereas tumors resistant to drugs or showing progressive growth gave the lowest average inhibition of target protein phosphorylation.

As mentioned above, in certain preferred methods and assay of the invention, RNA (such as mRNA) expression is not assessed.

A wide variety of cancer tumors may be assessed for therapeutic treatment in accordance with the invention. For instance, both solid tumors and disseminated tumors such as leukemia cells may be assessed. Specific tumors that may be assessed include e.g. cancer cells from a mammal such as a human and from the subject's brain, lung, ovary, breast, renal, pancreas, bladder, kidney, liver, testes, colon, or other cancer cells such as melanoma cells.

A variety of therapeutic agents also may be assessed in accordance with assays and methods of the invention to evaluate the effectiveness of the agent against a particular tumor. In particular, one or more of the following therapeutic agents may be evaluated for effectiveness against a particular tumor in accordance with methods and assays of the invention:

abarelix; aldesleukin; Aldesleukin; Alemtuzumab; alitretinoin; allopurinol; altretamine; amifostine; anastrozole; arsenic trioxide; asparaginase; azacitidine; BCG Live; bevacuzimab; bexarotene; bexarotene; bleomycin; bortezomib; busulfan; calusterone; capecitabine; carboplatin; carmustine; celecoxib; cetuximab; chlorambucil; cisplatin; cladribine; clofarabine; cyclophosphamide; cytarabine; dacarbazine; Darbepoetin alfa; daunorubicin; decitabine; Denileukin diftitox; dexrazoxane; docetaxel; Dromostanolone; doxorubicin; Elliott's B Solution; epirubicin; Epoetin alfa; erlotinib; estramustine; etoposide phosphate; etoposide; exemestane; Filgrastim; floxuridine; fludarabine; fluorouracil, 5-FU; fulvestrant; gefitinib; gemcitabine; gemtuzumab ozogamicin; goserelin; histrelin; hydroxyurea; Ibritumomab Tiuxetan; idarubicin; ifosfamide; imatinib; interferoninterferon; irinotecan; lenalidomide; letrozole; leucovorin; Leuprolide Acetate; levamisole; lomustine; meclorethamine; megestrol; melphalan, L-PAM; mercaptopurine, 6-MP; mesna; methotrexate; methoxsalen; mitomycin C; mitotane; mitoxantrone; nandrolone phenpropionate; nelarabine; Nofetumomab; Oprelvekin; oxaliplatin; paclitaxel; palifermin; pamidronate; pegademase; pegaspargase; Pegfilgrastim; pemetrexed; pentostatin; pipobroman; plicamycin, mithramycin; porfimer; procarbazine; quinacrine; Rasburicase; Rituximab; sargramostim; sorafenib; streptozocin; sunitinib; talc; tamoxifen; temozolomide; teniposide, VM-26; testolactone; thioguanine, 6-TG; thiotepa; topotecan; toremifene; Tositumomab; Tositumomab/I-131 tositumomab; Trastuzumab; tretinoin, ATRA; Uracil Mustard; valrubicin; vinblastine; zoledronate; and zoledronic acid.

Additional therapeutic agents that may be evaluated for effectiveness against specific tumors in accordance with methods and assays of the invention include, but are not limited to, the following described below.

Other examples of anti-cancer drugs that may be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elformithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

One or more chemotherapeutic or metabolic agents may also be selected from the group consisting of: Panitumumab; Erbitux; Temsiroliumus; Avastin; Tykerb; Herceptin; and Sutent.

Obesity and type 2 diabetes are the most prevalent and serious metabolic diseases; they affect more than 50% of adults in the USA. These conditions are associated with a chronic inflammatory response characterized by abnormal cytokine production, increased

acute-phase reactants and other stress-induced molecules. Many of these alterations are initiated and to reside within adipose tissue. Elevated production of tumor necrosis factor by adipose tissue decreases sensitivity to insulin. Several lines of evidence suggest that dysregulation of signaling pathways involving JNK, PI3K/AKT/GSK3, MEK/ERK are causally linked to aberrant metabolic control in obesity and insulin resistance in type 2 diabetes.

In vivo and ex vivo monitoring of tissue response obtained by fat pad biopsy can also be potentially used to assess the effect of hormones, such as insulin, and other cytokines in metabolic diseases such as obesity and type 2 diabetes to determine patients' sensitivity and resistance to therapeutic and preventive applications.

A significant challenge to developing ex vivo assays to assess the efficacy of targeted therapeutics arises from the difficulties relating to the selection of appropriate endpoints of drug sensitivity. Growth inhibition has traditionally been utilized in ex vivo assays to test tumor sensitivity to chemotherapeutic agents. However, this approach has been hampered by poor growth of tumor cells under assay conditions, which leads to significant problems in data interpretation. Such ex vivo growth of tumor cells and growth inhibition assessments are not employed in preferred assays and methods of the invention.

Therefore, the anticancer drug development paradigm will require the development of laboratory assays that can accurately measure the drug effect on the target in the clinic. For example for therapeutics blockading EGFR and its downstream signaling, techniques that permit the assessment of the phosphorylation-state of the target proteins in tumor tissues may be helpful in selecting optimal therapeutic agents and dosages in a clinical setting.

Having demonstrated that fine needle aspiration (FNAB) of tumor tissue yields enriched neoplastic cell populations, it was also then tested whether or not cancer cells obtained by FNAB can be used to determine sensitivity of tumor cells to targeted therapeutics ex vivo. Since growth inhibition may not be the best parameter to assess tumor cell sensitivity to targeted therapeutics, it was aimed to test tumor cell response at the biochemical level by analyzing therapy-mediated changes such as phosphorylation and/or acetylation status and/or expression levels of the target protein(s).

Also tested was the use of fat pad biopsy. Taking a series of repeat biopsies or fine needle aspirates of a tumor and adipose tissue during the course of therapy can provide information about treatment-induced changes in expression and activation of signaling and metabolic proteins and help monitor patient response to therapy. It is expected that this approach will also further our understanding of the molecular mechanisms that determine a patient's response or resistance to therapy in metabolic and neoplastic diseases, may facilitate investigation of molecular biology of disease response, and may provide useful information towards the development of new therapeutic and preventive agents.

As discussed above, in one aspect, a novel pharmacodynamic system has been developed for drug testing, which may suitably include use of a patient's tumor tissue obtained at the time of cancer resection.

Methods and assays of the invention have been significantly validated. In additional to the human patient studies as discussed above, tumors were heterotransplanted in athymic mice, expanded to numbers suitable for the evaluation of multiple treatments and then tested with different targeted drugs.

The following non-limiting examples are illustrative of the invention. All documents mentioned herein are incorporated herein by reference.

Materials and Methods

The following materials and methods were employed in the below examples.

Drugs

For the ex vivo studies, stock solutions of erlotinib (OSI-774, TARCEVA OSI Pharmaceuticals, Melville, N.Y.) were prepared in dimethyl sulfoxide (DMSO). Temsirolimus (CCI-779, Wyeth Research, Collegeville, Pa.) was prepared in 100% ethanol. Both agents were stored at −20° C. For the in vivo (xenograft) studies, drugs were prepared as follows: Erlotinib was dissolved in 10% DMSO, 10% pluronic and 80% PBS. Temsirolimus, was dissolved in 10% ethanol, 10% pluronic and 80% PBS. All drugs were freshly prepared, and used at an injection volume of 0.2 ml/20 g body weight. Drug doses and treatment schedules were based on previous studies (17, 18).

Gefitinib was provided by AstraZeneca (Wilmington, Del.). CI-1040 was a kindly gift from Pfizer (Ann Arbor, Mich.). Stock solutions were prepared in dimethyl sulfoxide (DMSO) and stored at −20° C. For in vivo studies, gefitinib was diluted in 5% (w/v) glucose solution, and CI1040 was prepared in a vehicle of 10% Cremophore EL (Sigma, St Louis, Mo.), 10% ethanol and 80% water. AG1478 and PD98059 were purchased from Calbiochem

Tumor Xenograft Development and Assessment

Four-week-old female athymic (nu+/nu+) mice were purchased from Harlan (Harlan Laboratories, Washington, D.C.). The research protocol was approved by the Johns Hopkins University Animal Care and Use Committee and animals were maintained in accordance to guidelines of the American Association of Laboratory Animal Care. Briefly, frozen pancreatic xenografts in first passage in mice, after being obtained from surgical specimens of patients undergoing pancreatic resection for adenocarcinoma at the Johns Hopkins Hospital were re-implanted subcutaneously in groups of 5 mice for each patient, with 2 small pieces per mouse (F2 generation). Tumors were allowed to grow to a size of 1.5 cm at which point they were harvested, divided into small ˜3×3×3 mm pieces, and transplanted to another 18-22 mice, with two tumors per mouse. Tumors from this second mouse-to-mouse passage were allowed to grow until reaching −200 mm³, at which time mice were randomized in the following three treatment groups, with 6 mice in each group: control (vehicle), erlotinib (50 mg/kg/day i.p.), and temsirolimus (20 mg/kg days 1-5 i.p.). Mice were monitored daily for signs of toxicity and were weighed three times per week. Tumor size was evaluated three times per week by caliper measurements using the following formula: tumor volume=[length×width²]/2 as previously reported (19). Relative tumor growth (RTG) was calculated by tumor growth of treated mice divided by tumor growth of control mice (T/C)×100. Experiments were terminated on day 28. Tumor response was defined as sensitive, when RTG was less than 50% on day 28.

Ex Vivo Studies

Tumor cells were collected by FNAB from the xenograft animals before the start of treatment using a sterile 25G short needle. Tumor samples were immediately transferred into 10 ml sterile prewarmed complete RPMI-1640 culture medium containing 10% FBS, penicillin (200 μg/ml), and streptomycin (200 μg/ml). Cells were incubated with 0.04% trypan blue (Sigma) dissolved in PBS (9.1 mM Na₂HPO4, 1.7 mM NaH₂PO4, and 150 mM NaCl, pH 7.4). The viable (membrane-intact) and dead cells were then counted using Neubauer hemocytometer and the total viable cell count was used to calculate final working volumes. Approximately 25,000 viable tumor cells were seeded into each well of a 6-well polypropylene microplate. Cells were treated in duplicates with vehicle (control), erlotinib (5 μM) or temsirolimus (1 μM) in a humidified 5% CO₂ incubator at 37° C. for 6 hours. No fibroblast and endothelial cell growth was observed. Following treatment, non-adherent and adherent cells (only a few, collected by scraping) were pooled together and centrifuged at 500×g for 5 min at 4° C. After washing with PBS, cells were lysed in 100 μL of ice-cold lysis buffer (50 mM Tris-HCl, 0.25M NaCl, 0.1% (v/v) Triton X-100, 1 mM EDTA, 50 mM NaF, and 0.1 mM Na₃VO₄, pH 7.4) containing protease and phosphatase inhibitors (Roche Molecular Biochemicals) and analyzed by Western blot.

In Vivo FNAB Studies

FNAB samples were collected from each animal before (day 0) and at the end (day 28) of treatment. The aspirated material was smeared onto clear glass slides and all smears were allowed to air dry and then stained with Diff-Quick stain (Baxter Healthcare, Miami, Fla.). Five air-dried and Diff-Quik (AD/DQ)-stained cytological smears were prepared from each tumor sample. The cellular composition of each aspirate was assessed by a certified staff cytopathologist (S.A.) at the Johns Hopkins Hospital under the microscope prior to protein extraction. To prepare whole cell lysates, the cells were collected from AD/DO slides by scraping into ice-cold buffer with protease and phosphatase inhibitors (Roche Molecular Biochemicals). Cell lysates were centrifuged in an Eppendorf microcentrifuge (14,000 rpm, 5 min) at 4° C., and the supernatants were used in Western blot experiments.

Tissue Preparation and Immunohistochemical Analysis

At the completion of the treatment course, xenografted tissues were harvested and fixed in formalin for 24 hrs. The fixed tissues were paraffin-embedded and cut in 0.5 micron sections onto positively-charged glass slides for immunohistochemical (IHC) labeling. Analysis was performed to determine the IHC pharmacodynamic effects of the drug in the targeted pathway. For IHC staining, slides were deparaffinized and rehydrated in graded concentrations of alcohol by standard techniques before antigen retrieval in citrate buffer pH 6.0 for 20 minutes. Next, the slides were cooled for 20 minutes before washing in 1×TBST (Dako Corp. Carpinteria, Calif.). All staining was performed using a DAKO Autostainer at room temperature. Slides were incubated in 3% H₂O₂ for 10 minutes, followed by the appropriate dilution of primary antibody for 60 minutes. Tris-HCl (0.2M, pH 7.5) (Quality Biological, Inc, Gaithersburg, Md.) was used as the antibody diluent solution. Slides were incubated in 3% H₂O₂ for 10 minutes, followed by the appropriate dilution of primary antibody for 60 minutes. Dilutions of antibodies used were as follows: Total ERK1/2 (Cell Signaling Technology, Beverly, Mass.) 1:25, p-ERK1/2 (Thr202/Tyr204) (Cell Signaling Technology, Beverly, Mass.) 1:50, p70S6K (Santa Cruz Biotechnology) 1:50, and pp 70S6K (Cell Signaling Technology, Beverly, Mass.) 1:50. Negative controls were incubated for 60 min with the antibody diluent solution (0.2M Tris-HCl, pH 7.5 from Quality Biological, Inc., Gaitersburg, Md.).

Western Blot Analysis

Protein concentrations obtained from FNAB samples were quantified before each experiment. Protein extracts (15 μg) were electrophoresed on a 10% (w/v) SDS-polyacrylamide gel. After electrotransfer to Immobilon-P membranes (Millipore), membranes were blocked at room temperature using SuperBlock (Pierce) for one hour. The primary antibodies were diluted at 1:1000 in 1:10 dilution of SuperBlock solution and the membranes were incubated with primary antibodies overnight at 4° C. The antibodies tested were phospho-ERK1/2 (Cell signal #9101), phospho-S6 Ribosomal Protein (Cell Signaling Technology, Beverly, Mass.) and total ERK1/2 (Cell Signaling Technology, Beverly, Mass.) and total S6 Ribosomal Protein (Cell Signaling Technology, Beverly, Mass.). The next day, the membranes were washed and incubated for 1 h at room temperature with horseradish peroxidase (HRP)-conjugated secondary antibodies, rabbit IgG-HRP (Santa Cruz Biotechnology), or mouse IgG-HRP (Santa Cruz Biotechnology) at a final dilution of 1:3000. Antibody binding was visualized using enhanced chemiluminescence (SuperSignal West Pico, Pierce) and autoradiography.

Cell Culture Experiments

T47D cells were maintained in Dulbecco's modified Eagle's medium (Life Technologies, Inc.) supplemented with 10% fetal calf serum and antibiotics (Life Technologies, Inc.). Prior to EGF (Sigma) stimulation, cells were starved for 24 h in serum-free medium.

Animal Studies

Four to 6-week-old female athymic (nu+/nu+) mice were purchased from Harlan (Harlan Laboratories, Washington, D.C.). The research protocol was approved by the Johns Hopkins University Animal Care Committee and animals were maintained in accordance with the guidelines of the American Association of Laboratory Animal Care. Mice were acclimatized for 1 week before injecting 2×106 HuCCT-1 human billiary tract cancer cells resuspended in 100 pl of MATRIGEL (Collaborative Biomedical Products, Bedford, Mass.) and 100 pI of PBS per mice. After 2 weeks when well-established tumors of 0.2 cm³ were detected, mice were randomly assigned in groups of 10 mice to receive the following treatments: gefitinib, 150 mg/Kg daily on days 1-5 and 8-12 administered by intraperitoneal injection; CI-1040 (150 mgr/Kg) twice a day on days 1-14 administered by oral gavage; combination of gefitinib+CI-1040 at the same doses and schedule of administration or; vehicle containing 0.15M CINa and 0.005% Pluronic. Mice were monitored daily for signs of toxicity and were weighed three times per week. Tumor size was evaluated three times per week by caliper measurements using the following formula: tumor volume=[length X width²]/2. Tumor growth inhibition was calculated by tumor volume of treated mice divided by tumor volume of control mice. Experiments were terminated on day 14.

Fine Needle Aspiration Technique

Fine needle aspirates were obtained with a 25G needle and 10 ml syringe, passing the needle through the tumor 10 times with application of 1-2 ml suction The aspirated material was expressed onto clear glass slides and smeared. All smears were allowed to air dry and then stained with Diff-Quik stain (Baxter Healthcare, Miami, Fla.). Five to ten AD/DQ-stained cytological smears were prepared from each tumor sample. The cellular composition of each aspirate was assessed by cytopathologists.

Western Blot Analysis and ELISA Assays

Total cell lysates were obtained from either cells cultured in vitro or from tumor FNAB samples. Protein extracts were resolved by 4-15% SDS-PAGE and probed with Rabbit anti-EGFR, anti-phospho-EGFR, anti-ERK1 and anti-phospho-ERK antibodies obtained from New England Biolabs (Beverly, Mass.). Immunoreactive proteins were visualized by enhanced chemiluminescence (Amersham International, United Kingdom). Total and phospho-ERK1/2 proteins were quantified by sandwich [LISA kits (BioSource International, Camarillo, Calif., USA) as described in the manufacturer's protocols. The reaction was read at 450 nm in an ELISA plate reader.

### EXAMPLES

### Example 1

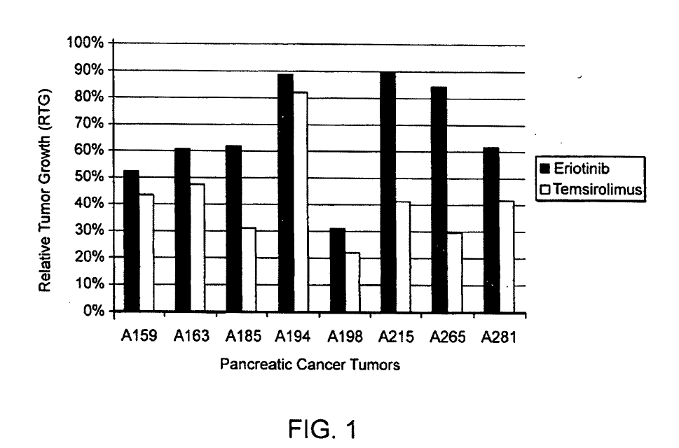

The efficacy of temsirolimus and erlotinib in the treatment of pancreatic cancer was tested in a series of mouse xenograft models of primary human pancreatic cancer. As shown in FIG. 1, in animals treated with temsirolimus, the relative tumor growth (RTG) ranged from 20% to 82% in eight pancreatic tumors. Except for tumor A194, all other tumors were sensitive to therapy, with less than 50% relative growth. In contrast, erlotinib was less active against the pancreatic cancer models with RTG between 30% and 90%. Only one tumor, A198, was sensitive to therapy, whereas the remaining seven tumors had greater than 50% RTG and were defined as resistant to erlotinib.

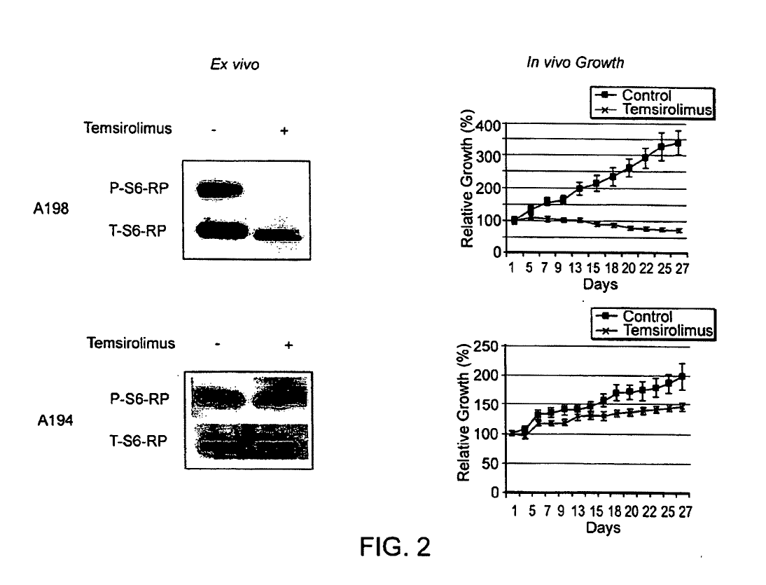

To test if tumor cells obtained by FNAB can be used in the ex vivo assays to predict tumor response in vivo, 25,000 viable cancer cells, as determined by trypan blue dye exclusion, were treated with erlotinib or temsirolimus for six hours, after which signal pathway inhibition was analyzed by Western blot. Under these cell culture conditions no fibroblast and endothelial cell growth was detected (data not shown). As shown in FIG. 2, treatment with temsirolimus ex vivo inhibited phosphorylation of S6-RP, an important regulatory kinase of the mTOR pathway, in cells collected from tumor A198, which are sensitive to therapy, but not in cells from tumor A194, which are resistant to anti-tumor effect of temsirolimus. Ex vivo treatment of cells did not affect the total levels of S6-RP protein (FIG. 2).

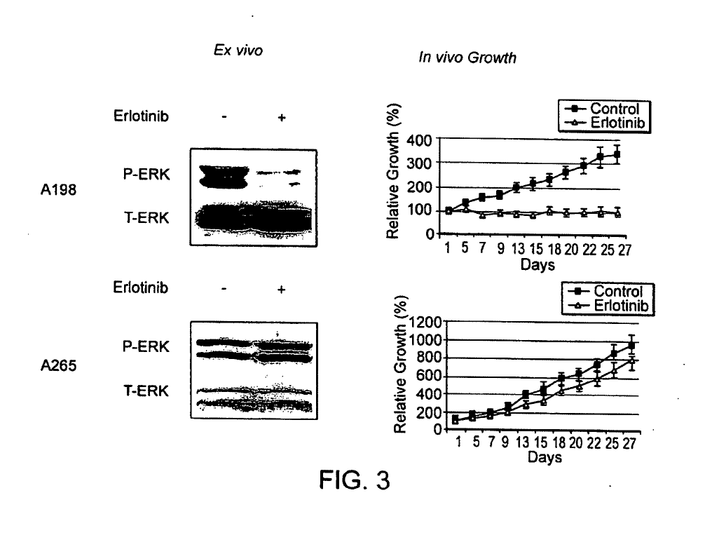

FIG. 3 illustrates that a dramatic inhibition was observed in phosphorylation of ERK1/2, a downstream effector of EGFR, in tumor cells derived from tumor A198, sensitive to erlotinib. However, erlotinib treatment failed to inhibit ERK1/2 phosphorylation in cells obtained from the resistant tumor A265. No changes were observed in the expression levels of total ERK1/2 in treated animals (FIG. 3). These data show that the ex vivo assays can predict tumor response in pancreatic tumors prior to in vivo treatment.

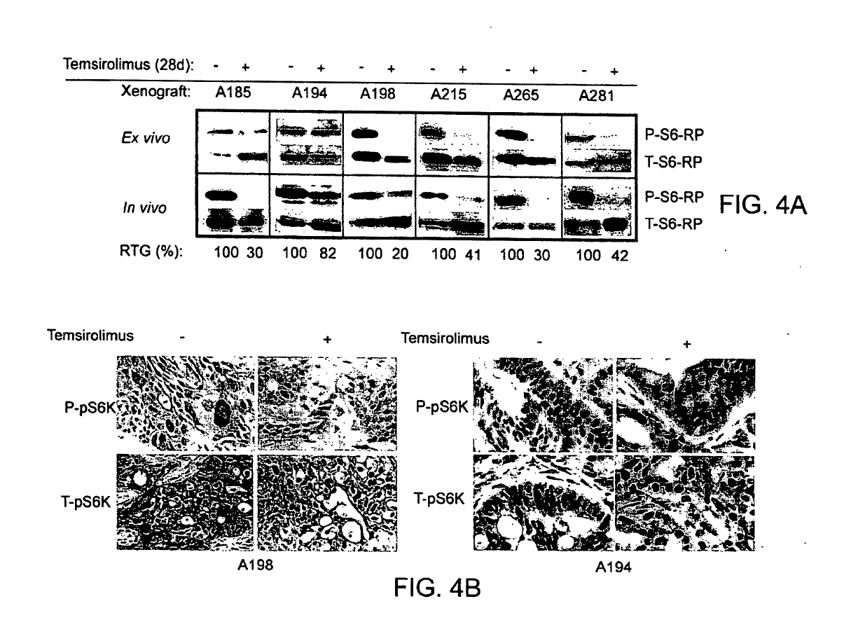

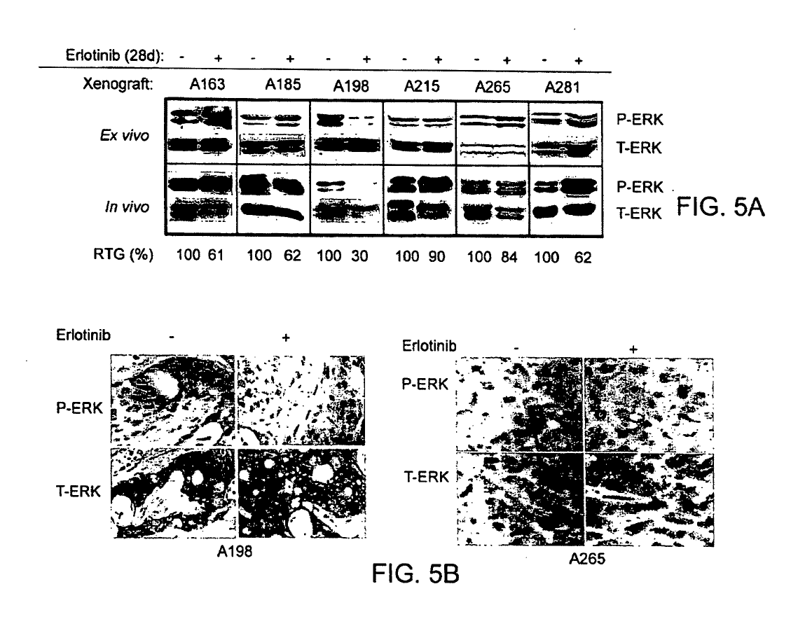

The reproducibility of these findings was further evaluated in the full panel of primary pancreatic xenografts. FIGS. 4A and 5A summarize the results of the ex vivo assays (upper panel) performed with all tumors and correlate drug-mediated inhibition of target protein activity with RTG. As shown in FIG. 4A, temsirolimus blocked S6-RP phosphorylation in all tumors sensitive to therapy ex vivo, but not in the tumor resistant to therapy. Consistent results were seen with erlotinib therapy, where the drug failed to inhibit ERK1/2 activation ex vivo in all resistant tumors, but did show inhibition of ERK1/2 in the one xenograft among the panel which had growth inhibition with erlotinib treatment. (FIG. 5A). To analyze the efficacy of temsirolimus and erlotinib in vivo, AD/DQ-stained smears were prepared from FNAB samples obtained from tumor tissue prior to initiation (day 0) and at the end (day 28) of treatment. Morphologic assessment of the cytologic smears demonstrated that, on average, 90% of the cells were neoplastic with some red blood cells and negligible amount of connective tissue fragments in the background. No significant apoptosis or necrosis was detected in tumors of control and drug treated animals (data not shown), indicating that targeted treatment had cytostatic rather than cytotoxic effect on tumor cells.

Following morphologic evaluation, whole cell extracts were prepared from AD/DQ-stained tumor FNAB samples and the expression levels of total and phosphorylated S6-RP and ERK1/2 proteins were determined on Western blot analysis. Overall, across the panel of xenografted primary pancreatic tumors, the pharmacodynamic effect of each drug ex vivo was concordant with in vivo target effect as well as with changes in tumor volume (FIGS. 4A and 5A).

To confirm the changes observed by FNAB analysis, immunohistochemical (IHC) staining of tumor tissue resected from vehicle and drug treated animals was performed (FIGS. 4B and 5B), and compared results with the Western blot data obtained from in vivo FNAB samples (FIGS. 4A and 5A). As illustrated in FIG. 4B, in tumor A198, which was sensitive to treatment, temsirolimus strongly decreased staining for the phosphorylated form of p70S6K (pS6K), a kinase in the mTOR pathway which regulates the activity of S6-RP. However, no effect was seen in tumor A194, which did not respond to temsirolimus in vivo. No significant changes were observed in total pS6K staining in treated tumors. These IHC results correlate with findings observed in Western blot analysis of FNAB specimens from the in vivo treated tumors (FIG. 4A). With erlotinib, however, the IHC results were rather inconclusive, partly due to the low intensity and focal staining pattern of phospho-ERK protein in both vehicle and erlotinib treated tumor samples (A198 and A265 xenografts depicted in FIG. 5B). This observation is likely due to the low sensitivity of the INC assays to detect phospho-ERK1/2 proteins in selected cases, rather than problems associated with the antibody used in these assays, since the same antibody was able to detect ERK1/2 expression in INC assays performed with other pancreatic tumor samples (data not shown). These results show that the FNAB-based approach is a viable alternative to conventional 1HC to evaluate morphological and molecular features of tumor cells in small tumor samples.

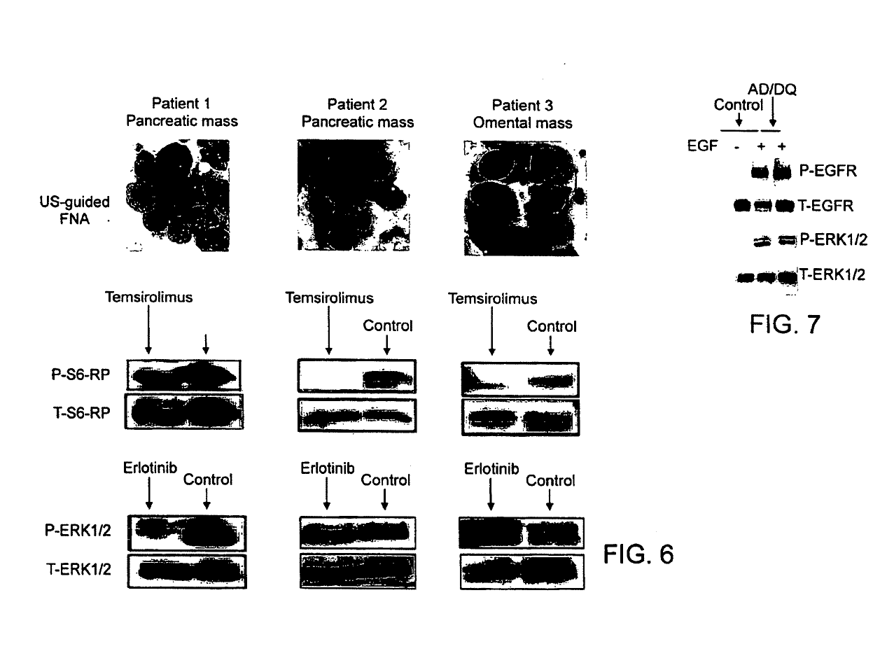

To determine whether standard clinical FNAB specimens provide adequately cellular tumor samples to perform ex vivo prediction assays, adenocarcinoma cells were collected by ultrasound or computer tomography-guided FNAB technique from pancreatic cancer patients during routine diagnostic procedures. Tumor cells were isolated by centrifugation from the needle rinse suspensions and treated with vehicle (control), erlotinib, or temsirolimus ex vivo for six hours. As illustrated in FIG. 6, adenocarcinoma cells with similar cytomorphological features showed variation in their responses to targeted therapeutics ex vivo. In cancer cells collected from patient 1, erlotinib dramatically blocked ERK1/2 phosphorylation, whereas temsirolimus only partially decreased S6-RP phosphorylation ex vivo. In tumor cells of patient 2 and 3, however, erlotinib did not effectively block ERK1/2 activity, whereas temsirolimus inhibited S6-RP phosphorylation. No inhibition was observed in the expression of total ERK1/2 and S6-RP proteins in drug treated cells (FIG. 6). Although these patients were not subsequently treated with the same agents to correlate ex vivo drug effects with clinical outcome, these results suggest that the ex vivo drug sensitivity assay employed in the preclinical model can be applied to clinical studies to predict patient response to targeted therapeutics prior to the initiation of treatment.

The advent of targeted therapy offers the potential of revolutionizing the treatment landscape for human cancer. In spite of encouraging early results in some settings, contemporary experience has begun to illuminate the relatively substantial challenges in the way of realizing the full promise of this new field. The specificity of this new class of agents is useful in terms of the possibility of identifying molecular markers of drug effects that might correlate with clinical outcomes.

If a given agent will be most effective against those tumors where its target is biologically critical, the next step is to develop clinically useful means of identifying that the biomarkers and agents. The development and validation of clinically relevant biomarkers of treatment efficacy will provide tools applicable to the enrichment of clinical trials and ultimately will provide individualized tailoring of therapy. At present, the dearth of reliable tools to rationalize treatment selection and monitor efficacy of a given regimen limits this realization.

Here, a novel pharmacodynamic assay in xenograft mouse models of human pancreatic cancer was evaluated, where tumors were obtained from primary clinical material. Prior studies show that these xenograft tumors maintain the features of the index tumor and are representative of the genetic heterogeneity of pancreatic cancer (Rubio et al.). The results of this study demonstrate that relatively small samples of tumor cells obtained by a well-established, minimally invasive diagnostic technique, FNAB, that can be used for reproducible assays to predict how tumors will respond to targeted anti-cancer agents prior to initiation of therapy. In a panel of xenografts there was a strong correlation between the pharmacodynamic effects of the drugs on activation of downstream targets in ex vivo conditions.

The resistance to erlotinib observed in the majority of the xenograft panel may be due, at least in part, to the high prevalence of activating mutations of K-ras, in pancreatic cancer (20). In fact, studies in lung cancer have demonstrated associations of K-ras mutation with resistance to EGFR targeted interventions (9, 21). Remarkably, however, primary human pancreatic adenocarcinomas evaluated in this study were highly sensitive to temsirolimus, supporting the importance of mTOR signaling to proliferation in pancreatic cancer (22). There was found no meaningful correlation between tumor responsiveness to erlotinib and the ability of the drug to inhibit EGFR phosphorylation (data not shown). This finding underscores the importance of validating candidate target markers as a prerequisite for pharmacodynamic-driven drug development.

The impediments to further development in this area may be organized under several broad themes. These relate to the selection and validation of endpoints or criteria of drug efficacy, the development of assays to evaluate those criteria, and tissue collection and sampling. Prospective determination of antibiotic sensitivity and resistance has been the standard of care in infectious diseases for many years. In contrast, due in part to the lack of reproducible predictive assays, treatment protocols for cancer patients have been driven by the taxonomy of tumor histology rather than a tumor's sensitivity to a given chemotherapeutic agent. Growth inhibition or cell death has been utilized in previous iterations of assays of sensitivity to conventional chemotherapeutic agents (23-29). However, due to poor tumor growth under assay conditions, labor-intensive and time-consuming methods and the use of uncertain criteria for defusing “sensitivity” or “resistance”, these assays have not gained wide clinical acceptance.

The majority of available studies attempting to correlate candidate biomarkers and response to targeted agents have been retrospective in nature and focused on static measurements of drug target expression and molecular evidence of dysregulation or activation in tissues (6-12). There are several limitations with this approach. First, the detection of target protein expression in archived pre-treatment samples may be inadequate to predict the activity of drugs, since the anticancer effect of a given agent may, in actuality, depend upon alterations in signaling both upstream and downstream of the target protein (9, 12). This biological complexity provides a point of departure to begin to understand the range of responses to targeted therapies among individual patients with apparently identical target protein expression levels (30, 31). Furthermore, the conventional approach does not account for potential changes in the biological status of targets over the natural history of an individual case. This is highlighted by recent demonstrations of spatial and temporal variation in EGFR expression following chemotherapy as well as in paired primary and metastatic colorectal cancers (32, 33).

The pharmacodynamic ability of a drug to inhibit the target pathway may be more important as a predictor of efficacy than the expression or activation of the target per se. Studies assessing pretreatment AKT activation as a predictor of response to anti-EGFR agents in lung cancer illustrate this point. Activated AKT has been reported to predict both positive and negative outcome in this setting (34-36). To the extent that these markers are evaluated as nodes along potential downstream pathways, such superficially contradictory results may be readily understood. In tumors dependent upon EGFR signaling through AKT, it stands to reason that AKT activation is a reasonable surrogate of susceptibility to the EGFR-targeted agent. In contrast, in a tumor dependent upon EGFR-independent pathways intersecting at AKT, activation of AKT may in fact represent uncoupling from upstream regulation by EGFR and portend resistance to its inhibitors. Taking this view, the challenge lies in identifying and characterizing the features of signaling nodes corresponding to biologically important pathway effectors.

A distinct advantage of targeted agents is the potential to develop assays specific to the molecular actions of the drug. For this purpose, S6-RP and ERK1/2 phosphorylation were used as two well validated and frequently used pharmacodynamic markers of mTOR and EGFR pathway blockade, respectively (37, 38). Described herein is an approach where drug inhibition of target pathway is a necessary, but not sufficient requirement for antitumor activity. The potential utility of assays such as those described herein may be greatest as a tool with high negative predictive value. The positive predictive value of pharmacodynamic assays of target inhibition may be tempered by cross-talking pathways downstream of the marker of interest and by factors such as tumor vasculature, metabolism and drug distribution to the tumor tissue.

The development of assays to predict tumor response to treatment is also hindered by problems of tissue acquisition. Previously explored chemo-sensitivity assays required relatively large tumor specimens (i.e., surgical biopsies), which necessitated general anesthesia for safe and reliable acquisition (39). FNAB is a minimally invasive, established diagnostic procedure that allows acquisition of enriched tumor cell populations to perform analytic molecular tests (40-46). The results presented herein demonstrated that sufficient protein quantities can be obtained from tumor FNAB samples to analyze the efficacy of targeted drugs ex vivo and in vivo. Given its safety, minimal morbidity and relative technical ease, FNAB is also suitable for serial sampling over the course of treatment to monitor therapy effect in vivo.

The performance of the FNAB studies appears quite feasible in xenograft tumors that, at the size sampled here, contain viable tumor cells with minimal necrotic contamination. An obvious question is whether similar materials can be obtained from patients' tumors. To address this concern, the feasibility of ex vivo assays in FNAB materials from diagnostic biopsy materials was evaluated. The results presented herein suggest that similar results as seen in the animal studies can be obtained from standard clinical materials. Future studies will determine the degree to which the results of such assays correspond to clinically observed treatment effects in humans.

In summary, in a novel in vivo model system for drug development and biomarker discovery in pancreatic cancer, FNAB-guided ex vivo drug assays appear to be a promising candidate tool to aid in the clinical development of targeted agents. Implementation of approaches such as those outlined herein in clinical studies may result in improved patient selection to maximize potential benefit while sparing patients unlikely to benefit from a given agent. Furthermore, this approach will provide a platform for the incorporation of multiple dynamic molecular analytical methods as well as the evaluation of more than one agent simultaneously. In the immediate term, this approach may offer a means of enriching clinical trials to better identify effective candidate regimens for patients with given tumor types. Ultimately, if validated in clinical trials, tools such as these may afford a means of tailoring the most efficient therapeutic regimen for individual patients.

### Example 2

It was tested if proteins prepared from air-dried and Diff-Quick stained (AD/DQ) cytologic samples can be used to analyze phosphorylation and expression levels of EGFR and ERK1/2 proteins by western blot (WESTERN BLOT) analysis. For this purpose, equal numbers of T-47D breast cancer cells were serum starved overnight and collected by scraping either before or after stimulation with EGF (100 ng/ml) for 15 min. Cell pellets were used to prepare air-dried cytologic smears on glass slides followed by Diff-Quick staining. Protein extracts were prepared from smear samples and total levels, as well as phosphorylated, EGFR and ERK1/2 proteins were analyzed by Western blot using 15 ug of total cell lysates. Phosphorylation status reflects the activation state of the protein, e.g. phosphorylated EGFR (P-EGFR) is signaling active EGFR. The results were compared to control cell extracts prepared directly from cells grown on culture plates. In control extracts prepared from EGF-treated T-47D cells, phospho-specific antibodies to EGFR and ERK I/2 showed increased phosphorylation of these proteins compared to EGF unstimulated cells (FIG. 7). In protein samples prepared from air-dried/DQ-stained T-47D smears, almost identical results were observed in expression and phosphorylation levels of EGFR and ERK1/2. Thus, these findings suggest that air-dried Diff Quick stained cytologic samples obtained from a patient's tumor may allow the analysis of the expression and phosphorylation pattern of EGFR signaling proteins in vivo.

Taken together, these results demonstrate that methods used in preparation of air-dried cytologic samples do not affect the quality of proteins for the analysis of the activation/phosphorylation pattern of EGFR signaling proteins using western blot analysis.

### Example 3

Western blot analysis of protein samples is a conventional method for phosphoprotein analysis. However, Western blot analysis is limited in throughput and quantitative precision, and also requires large sample amounts. The enzyme linked immunosorbent assay (ELISA) offers an alternative to Western blot that has higher throughput and increased sensitivity. Therefore, it was tested if quantitative ELISA assays can be applied to cytologic samples to increase the assay sensitivity to measure the expression levels and activation status of specific signaling pathways. As a model system, two different ERK1/2 ELISA assays were used: (1) colorimetric total, which recognizes proteins independent of their phosphorylation (Biosource International, KHO0081) and (2) phosphospecific, which recognizes only the phosphorylated (activated) state of signaling components (Biosource International, KHO0091) to analyze the expression and phosphorylation of ERK1/2 proteins, respectively, in air-dried T-47D cell smears.

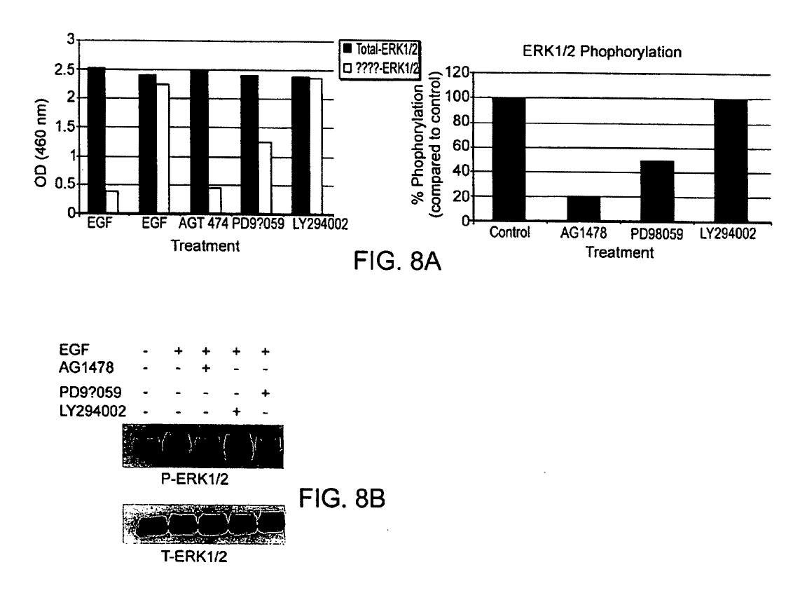

First, the linearity of the ELISA assays was determined by using various protein amounts (0.5-20 ug) obtained from air dried T-47D cytologic samples. Briefly, cell extracts were prepared in 0.1% SDS lysis buffer, boiled, and analyzed in the ERK1/2 [pTpY185/187] phosphor- and total ELISA assays. Protein amounts in the range of 0.5 to 5 ug yield the most accurate and linear determination of total and phosphorylated ERK proteins. Next, it was tested whether ELISA assays can detect treatment-mediated changes in the phosphorylation status of ERK1/2 in air-dried smears. For this purpose T-47D cells were stimulated with EGF in the presence or absence of various inhibitors to block EGF-induced activation of EGFR, ERK and AKT proteins. The expression levels and phosphorylation status of ERK1/2 were analyzed in ELISA assays by using 1 ug microgram of protein extracts prepared from air-dried smears. The OD values obtained by an ELISA plate reader from control cells and treated cells at 450 nm were quantified with the aid of internal total- and phospho-ERK1/2 standard proteins in parallel assays. Phospho-ELISA results were normalized for the content of ERK1/2, as determined by total ERK1/2 ELISA. As shown in FIG. 8A, treatment of T-47D cells with EGF led to a dramatic increase in the phosphorylation of ERK1/2, which was significantly (80%) and partially (50%) inhibited by an EGFR inhibitor AG1478 (0.5 uM) (Calbiochem, 658548) and by an MEK/ERK inhibitor PD98059 (20 uM) (Biosource International, PHZ1164), respectively. As expected, addition of a PI3KJAKT inhibitor LY294002 (10 uM) (Biosource International, PHZ 1144) did not have inhibitory effect on EGF-induced ERK1/2 activity. These results were corroborated by Western blot analysis (FIG. 8B), which demonstrate that the use of less than one-tenth of the amount of total cellular extracts required to detect ERK1/2 on Western blot is sufficient to quantitatively analyze treatment-mediated changes in the phosphorylation of p42/p44 ERK1/2 in cytologic samples.

Taken together, the results show that air-dried cytologic samples yield high quality proteins that are useful to study the activity of signal transduction pathways by determining the phosphorylation status of enzymes involved in cell growth and survival.

### Example 4

To explore the feasibility of implementing this method in in vivo studies it was next tested in mouse xenografts whether FNAB material obtained from tumor tissue can be utilized to monitor and predict therapy response in vivo. For this purpose HUCCT-1 cholangiocarcinoma cells were used (kindly provided by Dr. Anirban Maitra, Johns Hopkins School of Medicine), which overexpress ERK1/2 proteins and exhibit constitutive activation of EGFR, to create a xenograft mouse model of human biliary carcinoma.

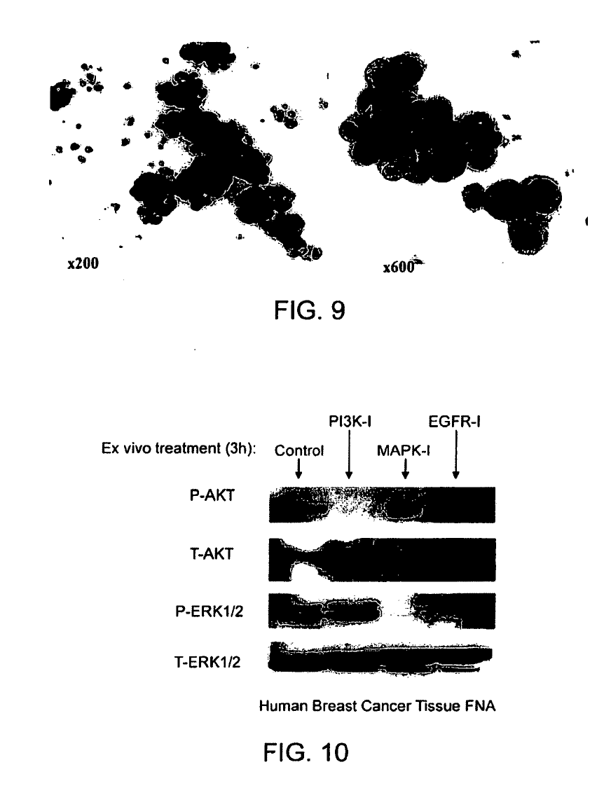

As shown in FIG. 9, FNAB samples yielded a nearly pure tumor cell population with some red blood cells and a negligible amount of connective tissue fragments in the background.

No significant apoptosis or necrosis was detected in control, ZD1839 and/or CI-1040 treated tumors, as cytologic and histologic preparations of tumor samples were evaluated microscopically (data not shown). After comparison with the histologic sections of the same tumors, it was determined that FNAB samples yielded adequate materials to represent the composition of HUCCT-1 tumor tissue.

### Example 5

Fine needle aspiration yielded up to 200 μg of total protein as determined using the BCA protein assay (Pierce) and bovine serum albumin as a standard. Protein extracts (15 μg) were added to a loading buffer boiled and electrophoresed on a 7 or 10% (w/v) polyacrylamide gel in the presence of SDS. Molecular weights of the immunoreactive proteins were estimated based upon the relative migration with colored molecular weight protein markers (Amersham Pharmacia Biotech). After electrotransfer to Immobilon-P membranes (Millipore), membranes were blocked at room temperature using SuperBlock (Pierce, #37516) for one hour. The primary antibodies were diluted at 1:1000 in 1:10 dilution of SuperBlock solution and the membranes were incubated with primary antibodies overnight at 4° C. Next day, the membranes were washed and incubated for 1 h at room temperature with horseradish peroxidase (HRP)-conjugated secondary antibodies, rabbit IgG-HRP (SC-2004), or mouse IgG-HRP (SC-2005) from Santa Cruz Biotechnology at a final dilution of 1:3000. After washing three times with Tris-buffered saline antibody binding was visualized using enhanced chemiluminescence (SuperSignal West Pico, Pierce) and autoradiography.

The expression levels and the phosphorylation status of EGFR, ERK1/2 and S6 ribosomal protein were determined on Western blot analysis. As shown in FIG. 10B, compared to control tumor samples treatment with ZD1839 completely abolished EGFR but not ERK1/2 phosphorylation. CCI-779 therapy, on the other hand, effectively blocked S6 ribosomal protein phosphorylation in vivo. No difference was observed in the protein levels of EGFR, ERK1/2 and S6 ribosomal protein between vehicle and drug treated animals.

### Example 6

To demonstrate that cancer cells obtained by tumor FNAB can be used to assess tumor response to therapy, human breast cancer cells were obtained by FNAB from tumor tissue surgically removed for therapeutic purposes. To obtain cancer cells cancer tumor tissue was sampled four times with a sterile 25G short needle and tumor samples were immediately transferred into 10 ml sterile prewarmed complete RPMI-1640 culture medium containing 10% fetal calf serum, penicillin (200 ug/ml) and streptomycin (200 ug/ml). After centrifugation and cells were resuspended and distributed in 6-well microculture plates and treated in duplicates with vehicle alone (DMSO control), an EGFR inhibitor AG1478 (0.5 uM) (Calbiochem, 658548), a MEK/ERK inhibitor PD98059 (20 uM) (Biosource International, PHZ 1164) or with a PI3K/AKT inhibitor LY294002 (10 uM) (Biosource International, PHZI 144) in a humidified 5% CO₂ incubator at 37° C. for 3 hours.

Following treatment nonadherent and adherent cells (collected by scraping) were pooled together in a 1.5 ml microcentrifuge tube and centrifuged at 500×g for 5 min at 4° C. After washing with PBS, cells were lysed in 100 μl of ice-cold lysis buffer and therapy-induced changes n the expression and phosphorylation levels of AKT and ERK1/2 were detected by Western blot. As illustrated in FIG. 4, MAPK and PI3K/AKT inhibitors effectively and selectively blocked phosphorylation of their target proteins, whereas the EGFR inhibitor AG1478 reduced the phosphorylation of both ERK1/2 and AKT proteins, as expected. No changes were observed in the total expression levels of these proteins upon treatment with the inhibitory compounds.

These results demonstrate that neoplastic cells obtained by tumor FNAB provide invaluable information to assess tumor response to targeted signal transduction inhibitors ex vivo.

Next, the data was validated using xenograft animal models. A xenograft mouse model of human pancreas cancer, Panc 265, was prepared from a primary human pancreas adenocarcinoma surgically resected for therapeutic purposes. Briefly, after resection fresh tumor tissue was immediately placed in sterile RPMI 1640 medium supplemented with 20% fetal bovine serum and 0.05% gentamicin. Tumor tissue was then cut into slices (5×5^{x}0.5-1 mm diameter) and implanted subcutaneously into nude mice. Tumor slices averaging 5×5×0.5-1 mm diameter from the patient, or 3×3×0.5-1 mm dia in serial passage were implanted subcutaneously into both flanks of nude mice. Tumors were removed under sterile conditions/laminar flow and reimplanted subcutaneously in groups of 5 mice. When the tumors on second passage of each group reached 1.5 cm, they were excised and cut into pieces of 3×3×3 mm, and transplanted on another 35-40 mice. On the third passage, the rate of successful growth was 86-95%. Overall, the architecture and characteristics of the original tumor were maintained during early passages in mice.

For the in vivo assessment experiments, treatment started when the mean tumor volumes reach approximately 200 mm. Drugs were prepared as follows: For in vivo studies, ZD1839 (AstraZeneca (Wilmington, Del.) was diluted in 5% (w/v) glucose solution. CCI-779 (Wyeth Research, Colleville, Pa.) was dissolved in 10% ethanol, 10% pluronic and 80% PBSA11 drugs were freshly prepared, and used at an injection volume of 0.2 ml/20 g body weight. Drug doses and treatment schedules were optimized in previous studies (Hidalgo et al unpublished results).

Animals were either untreated or treated with ZD1839: 150 mg/Kg daily or CCI-779: 20 mg/kg days 1-5 by intraperitoneal injection

To obtain cancer cells, each animal tumor was sampled as described above. After collection of tumor FNAB samples, xenograft animals were subsequently treated with ZD1839 or CCI-779 for 28 days and tumor volumes were determined as described above. At day 7 of therapy, AD/DQ slides were prepared from tumor FNAB samples to retrospectively correlate the results of in vivo and ex vivo chemosentitivty tests from the same xenograft animal.

To determine viability of tumor cells obtained by FNAB, trypan blue dye exclusion assay was performed. The viability of tumor cells obtained by tumor FNAB from control animals was over 95%. Approximately 75,000 tumor cells were seeded into each well of a 6-well polypropylene microplate. Cells were treated in duplicates with vehicle (control) or with ZD1839 (5 uM) or CCI-779 (1 uM) in a humidified 5% CO₂ incubator at 37° C. for different periods to determine the optimal treatment time to analyze drug effects. Depending on the tumor type, approximately 10 to 30% of the cells showed adhesion to culture plate after three hours of incubation, whereas the adhesion rate was about 30-75% after 16 hours.

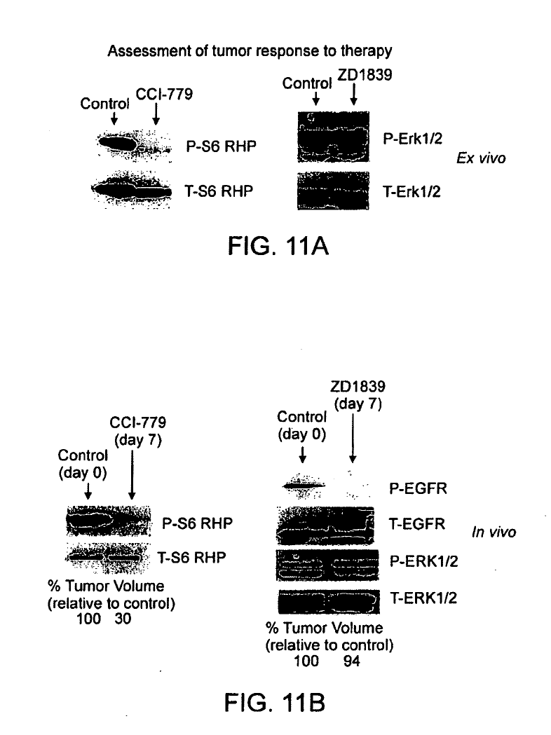

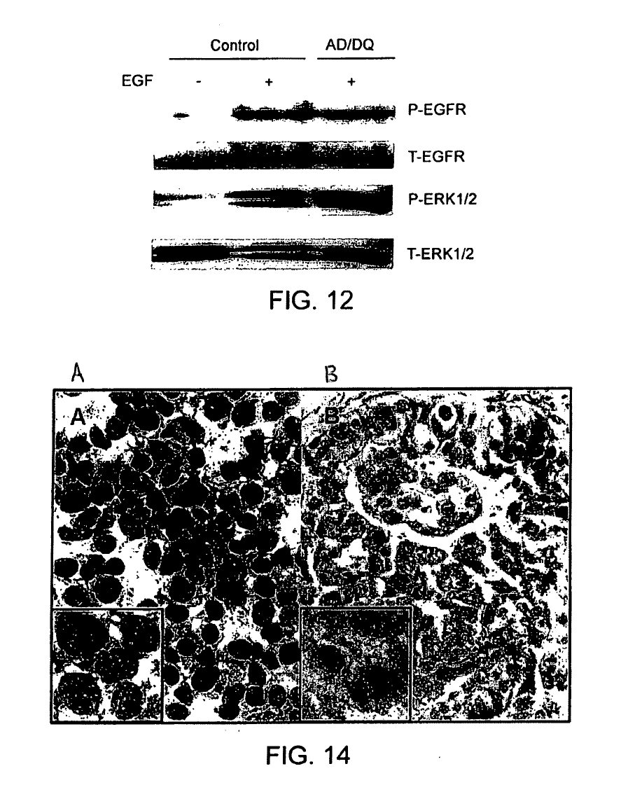

After the treatment cells were collected, protein extracts were prepared and Western blot analysis was performed as described above. The ex vivo effect of ZD 1839 and CCI-779 on phosphorylation of ERK1/2 or S6 ribosomal proteins, respectively, was correlated with the in vivo data gathered from the FNAB smears prepared at day 7 of treatment. As shown in FIG. 11A (upper panel), ZD1839 failed to block ERKI/2 phosphorylation, whereas CCI-779 treatment successfully inhibited the activity of its target protein ex vivo. No changes were observed in total expression levels of ERK1/2 and S6 ribosomal protein (S6-RBP), as shown in FIG. 11A (lower panel).

The degree of inhibition in the target protein phosphorylation by ZD1839 and CCI-779 ex vivo showed close correlation with tumor sensitivity in vivo (FIG. 4B, lower panel), where CCI-779 strongly inhibited target protein phosphorylation and tumor growth (70%), whereas ZD1839 treatment failed to block ERK1/2 activity and to achieve growth inhibition. Additionally, an increase in the treatment time from 6 to 16 hours did not cause any additional changes in protein phosphorylation of target proteins ex vivo (data not shown).

These data confirms that the FNAB-based in vivo sensitivity approach can assess tumor response in vivo. Furthermore, the results described here indicates that the FNAB-based ex vivo sensitivity assay can offer new opportunities to predict tumor response to targeted therapeutics in vivo.

### Example 7

To determine whether cellular proteins obtained by tumor FNAB can be used to assess the efficacy of targeted therapeutics, it was first tested if fixation and staining methods commonly used in the preparation of cytologic samples adversely affect detection of phosphorylation status of cellular signaling proteins. For this purpose, T47D cell lines were used in controlled studies.

Equal numbers of T47D cells were serum starved overnight and cultured in the presence or absence of epidermal growth factor (EGF) (100 ng/ml) for 15 min. Cells were harvested by scraping and cell pellets were used to prepare either control protein extracts or to prepare air-dried cytologic smears on glass slides. Smear samples were allowed to air dry, stained using Diff Quik (Wright's) stain and examined by light microscopy to confirm the presence of tumor cells. Then, protein extracts were prepared by scraping cells off the stained slides in lysis buffer and expression levels as well as phosphorylation status of EGFR and ERK1/2 proteins were analyzed on Western blot by using 15 p.g of total cell lysates. The results obtained from smear samples were compared to control cell extracts.

As illustrated in FIG. 12, lanes 1 and 2, in control extracts prepared directly from EGF-treated T47D cells, phospho-specific antibodies detected increased phosphorylation of EGFR and ERK1/2 compared to EGF unstimulated cells. EGF treatment did not cause any changes in the expression levels of these proteins as shown by antibodies recognizing EGFR and ERK1/2 independently from their phosphorylation state (FIG. 12). The expression and phosphorylation patterns of EGFR and ERK1/2 proteins in tumor lysates isolated from AD/DQ-stained T47D smears were almost identical to those observed with control extracts (FIG. 12, lane 3). As compared with other fixation and staining methods utilizing ethanol-containing solutions, commonly used in preparation of cytologic samples, AD/DQ-stained cytologic samples yielded superior quality and quantity of proteins to analyze activation/phosphorylation status of signaling proteins on Western blot (data not shown).

These results indicate that changes in the expression and phosphorylation profiles of EGFR signaling proteins in response to targeted therapies may also be analyzed in cell extract prepared from AD/DQ-stained smears of patient's tumor FNAB samples in vivo.

### Example 8

It was next tested if quantitative ELISA assays can be applied to cytologic samples to increase the assay sensitivity to measure the expression levels and activation status of specific signaling pathways. As a model system, colorimetric total- (recognizes proteins independent of their phosphorylation) and phosphor-specific (recognizes only the phosphorylated (activated) state of signaling components) ERK1/2 ELISA assays were used to analyze the expression and phosphorylation of ERK1/2 proteins, respectively.

First the linearity of these assays was determined by using various protein amounts (0.5 to 20 μg) obtained from AD/DQ-stained T47D cytologic smears. The results showed that protein concentrations in the range of 0.5 to 5 pg yield the most accurate and linear determination of total and phosphorylated ERK1/2 levels.

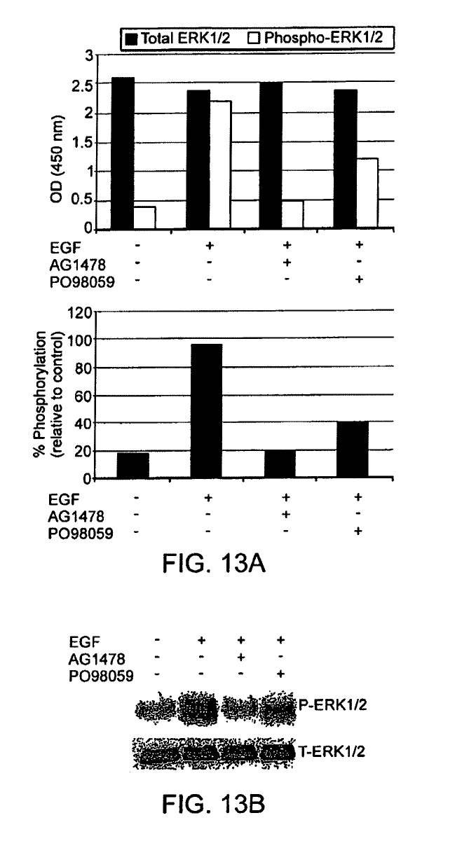

Next, it was tested whether ELISA assays can detect treatment-mediated changes in the phosphorylation status of ERK1/2 in AD/DQ-stained smears. For this purpose, T47D cells were stimulated with EGF in the presence or absence of various inhibitors of EGFR and MEK/ERK pathways. After treatment, extracts were prepared from AD/DQ-stained smears of T47D cells and expression levels as well as phosphorylation status of ERK1/2 were analyzed in ELISA assays by using 1 μg of whole cell lysates. The OD values obtained from control and treated cells by an ELISA plate reader at 450 nm were quantified with the aid of internal total- and phospho-ERK1/2 standard proteins in parallel assays. Phospho-ELISA results were normalized for the total contents of ERK1/2, determined by total ERK1/2 ELISA. As shown in FIG. 13A, stimulation of cells with EGF led to an increase in ERK1/2 phosphorylation (upper graph) and this increase was inhibited 80% and 60% by prior incubation of cells with EGFR and MEK/ERK inhibitors AG1478 (0.5 μM) and PD98059 (20 μM), respectively (lower graph). The results obtained by quantitative ELISA were corroborated by Western blot analysis (FIG. 13B), which demonstrate that the use of less than one-tenth of the amount of total cellular extracts required to detect ERK1/2 on Western blot is sufficient to quantitatively analyze treatment-mediated changes in the phosphorylation of p42/p44 ERK1/2 in cytologic samples.

### Example 9

FNAB samples yield enriched tumor cell populations to study EGFR signaling in vivo. The results shown above demonstrated that AD/DQ-stained cytologic samples yield high quality proteins to study the activity of signal transduction pathways by determining the phosphorylation status of enzymes involved in cell growth. To explore the feasibility of implementing this method in in vivo studies, mouse xenografts were next employed to test whether FNAB material obtained from tumor tissue can be utilized to monitor and predict therapy response in vivo. For this purpose HuCCT-1 cholangiocarcinoma cells were used to create a xenograft mouse model of human cholangiocarcinoma. Cells were injected into athymic nude mice and following the formation of tumors, animals were treated with gefitinib and CI-1040 alone or in combinations for 14 days. Tumor volumes were measured and compared with tumors from animals that received drug vehicle alone.

FNAB samples were obtained from tumor tissue and AD/DQ-stained smears were prepared. Morphologic assessment of the cytologic smears demonstrated that, on average, 90% of the cells were neoplastic with some red blood cells and negligible amount of connective tissue fragments in the background (FIG. 14A). Through comparison with the histologic sections of the same tumors (FIG. 14B), it is shown that FNAB samples yielded adequate materials to represent the composition of HuCCT-1 tumor tissue.

### Example 10

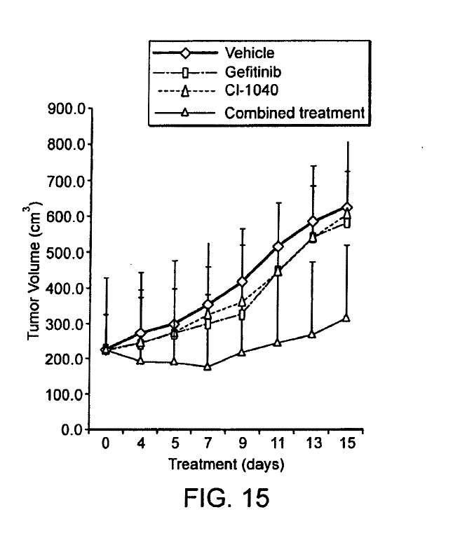

Combination of ZD1839 and CI-1040 therapy is required to block tumor growth in HUCCT-1 xenograft animals. As shown in FIG. 15, neither gefitinib nor CI-1040 alone inhibited tumor growth and only co-treatment with these two agents was effective against HuCCT-1 tumors. Tumor growth was inhibited by approximately 60% with gefitinib and CI-1040 combination therapy over the 14-day treatment period. By contrast, treatment with gefitinib or CI-1040 alone caused only 4% and 11% decrease in tumor volume, respectively.

These data indicate that inhibition of EGFR activity alone is not sufficient to block tumor growth and imply that blockade of both EGFR and ERK1/2 activity is necessary to achieve tumor growth inhibition.

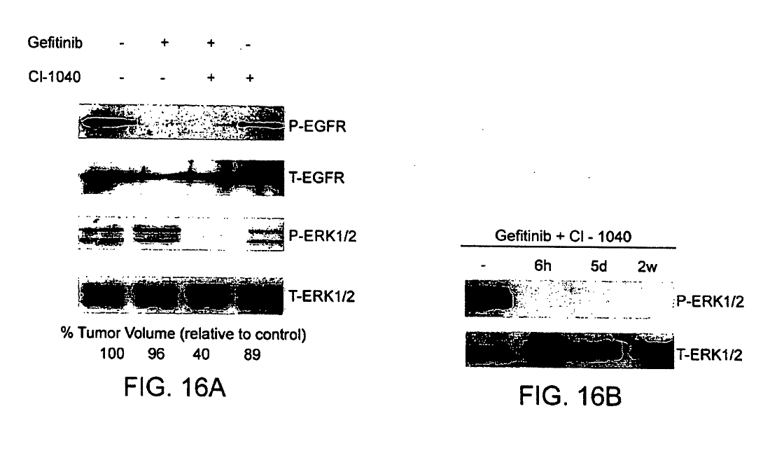

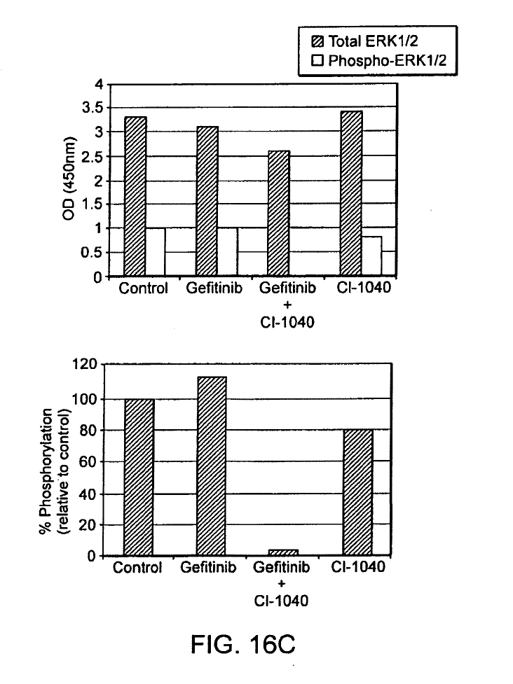

Tumor FNAB samples provide adequate quality proteins to analyze therapy-mediated changes in the activity of EGFR and ERK1/2 in vivo. To better understand the molecular mechanism by which only the combination but not individual treatment with gefitinib and CI-1040 causes tumor inhibition, the steady-state levels of EGFR and ERK1/2 kinases were examined in tumor FNAB samples collected from control and drug-treated mice. Following morphologic evaluation whole cell extracts were prepared from AD/DQ-stained tumor FNAB samples, which on average yielded 100 μg of total cellular proteins. FIG. 16A shows that EGFR and ERK1/2 were constitutively activated in the HuCCT-1 tumors as measured by immunoblotting of tumor lysates with phospho-EGFR and phospho-ERK1/2 antibodies, respectively. Samples from animals treated with gefitinib showed complete inhibition of EGFR but not ERK1/2 activity, indicating that the elevated steady-state levels of ERK activity in HuCCT-1 cells are not sustained predominantly through activation of EGFR. Interestingly, only combination treatment with gefitinib and CI-1040 dramatically lowered level of activation of ERK1/2 proteins, while treatment of animals with CI-1040 alone caused only a slight inhibition in ERK1/2 activity. No significant difference was observed in the protein levels of EGFR and ERK1/2 proteins between vehicle and drug treated animals (FIG. 16A). Correlation with therapy-mediated changes in tumor size revealed that the reduction in HuCCT-1 tumor growth rates coincides with inhibition of constitutive ERK1/2 but not EGFR activation, providing molecular evidence to explain why treatment with both gefitinib and CI-1040 is required to block growth of HuCCT-1 tumors.

These results demonstrate that tumor FNAB samples yield adequate amount and quality of cellular proteins to assess therapy-mediated changes in the activity and expression levels of EGFR signaling molecules in vivo.

### Example 11

Serial FNAB sampling permits monitoring and prediction of treatment response to EGFR and MEK inhibitors in vivo. Next, whether FNAB samples obtained from tumor tissue at the early stage of therapy can be used to predict tumor response was examined. For this purpose, FNAB was performed on the same animal's tumor before, during (6 hours and 5 days) and at the end (2 weeks) of gefitinib and/or CI-1040 therapy. Expression and phosphorylation levels of ERK 1/2 were analyzed on Western blot by using extracts prepared from AD/DQ-stained FNAB samples. As shown in FIG. 16B (upper panel), as early as 6 h after the first administration of gefitinib and CI-1040 a dramatic loss was observed in the ERK1/2 activity, which was sustained over the course of treatment for two weeks. Consistent with data described above, neither of these agents alone caused inhibition in ERK1/2 phosphorylation after 6 h or 5 d of treatment (data not shown). These effects were not due to alteration of ERK1/2 expression in treated animals, since no change was observed in total levels of ERK1/2 proteins in tumor samples obtained before and after the treatment (FIG. 5B, lower panel).

These data demonstrate that FNAB sampling at the early stage of therapy permits prediction of tumor response in vivo.

Combination of FNAB with a quantitative ELISA increases the sensitivity and accuracy to detect therapy-mediated changes in ERK1/2 activity in vivo. Western blot analysis of protein samples is a conventional method for phosphoprotein analysis, but is limited in throughput, quantitative precision and requires large sample amounts. ELISA assays offer alternatives to Western blot with higher throughput and increased sensitivity. Having established above that air-dried cytologic samples can successfully be used in ELISA assays to analyze ERK1/2 phosphorylation in vitro, it was next tested if this approach can be utilized to increase assay sensitivity and to quantify treatment-mediated changes in the phosphorylation of ERK1/2 in HuCCT-1 xenograft animals in vivo. For this purpose the tumor FNAB samples, which have been analyzed on Western blot above (FIG. 16A), were used to assess the expression and phosphorylation of ERK1/2 proteins by the total and phospho-specific ERK1/2 ELISA assays, respectively.

As shown in FIG. 16C, upper graph, treatment of animals with combination of gefitinib and CI-1040 significantly decreased the phosphorylation levels of ERK1/2, as detected by phospho-ERK1/2 ELISA, whereas neither gefitinib nor CI-1040 treatment alone caused any significant change in ERK1/2 phosphorylation. The amounts of phosphorylated ERK1/2 were normalized for the total contents of these proteins in each sample group and therapy-mediated changes in their phosphorylation status were quantified. FIG. 16C, lower graph, illustrates that treatment of animals with combination of gefitinib and CI-1040 caused a 98% inhibition of ERK1/2 phosphorylation, whereas, gefitinib or CI-1040 alone resulted in 17% increase and 19% decrease, respectively. These results are consistent with the Western blot data, shown above (FIG. 16A), and demonstrate that the use of less than 1 p.g of whole cell lysate is sufficient to quantitatively analyze therapy-induced changes in the enzymatic activity of ERK1/2 by ELISA in tumor FNAB preparations.

### Example 12

Several human cancer patients were evaluated in accordance with assays of the invention. Tumor cells were obtained from the patients by fine needle aspiration or endoscopic tumor biopsies. The susceptibility of the tumor cells to Iressa were evaluated ex-vivo prior to the commencement of chemotherapy and during the course of treatment. Modification and expression of target proteins were assessed. Results are set forth in FIG. 17 A through H.

### Example 13

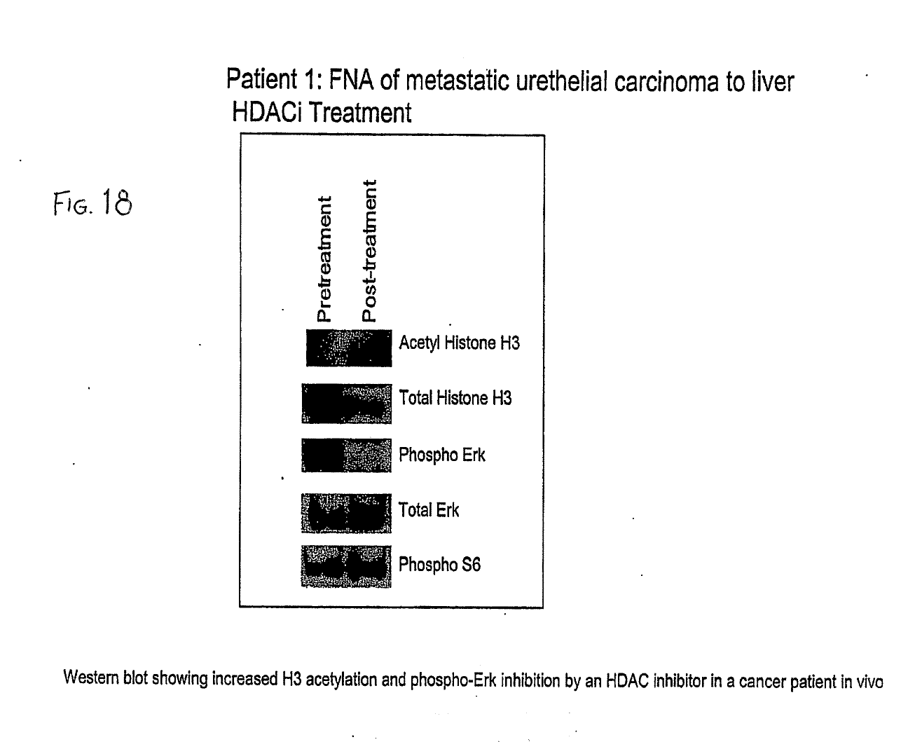

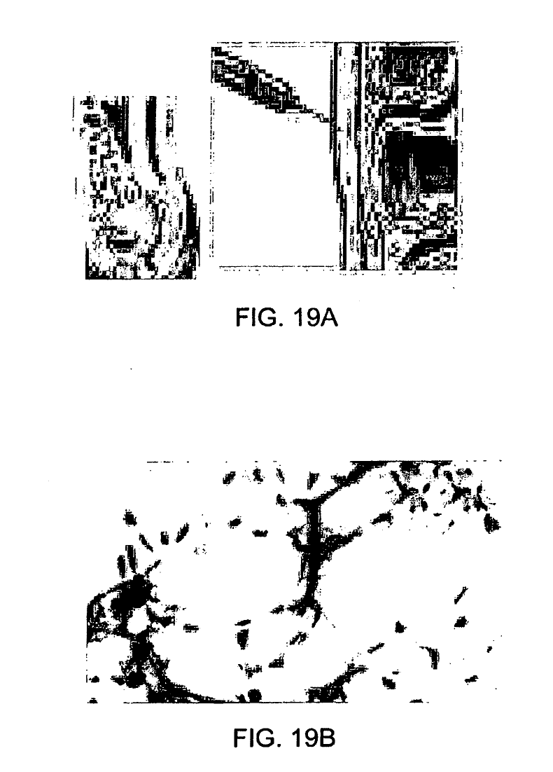

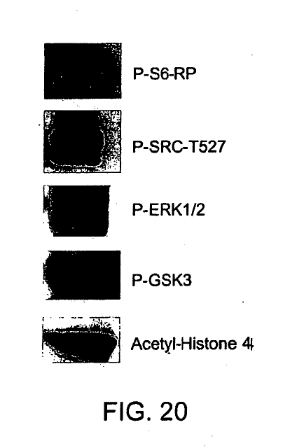

A fine needle aspiration was made from a metastatic urethelial carcinoma. The tumor sample was assessed with an HDAC inhibitor, including through use of a Western blot analysis of H3 acetylation and phopho-ERK inhibition as shown in FIG. 18.

### Example 14

Fat pad biopsy is a relatively noninvasive, economical, and fast procedure and commonly used to analyze amyloid deposition by Congo Red staining in routine pathology practice. However, phospho-proteomic analysis of cellular signaling in fat pad biopsies has never been explored before. Recently, it was shown that fat pad biopsy materials yield high quality protein to assess the phosphorylation status of key signaling pathway elements. Our results demonstrated for the first time that proteins isolated from fresh and air-dried and diff quick-stained fat pad biopsy smears samples allow detection of phosphorylation of signaling proteins such as SRC, AKT, ERK, S6-Ribosomal protein (S6-RP), and GSK3 involved in growth and differentiation signaling pathways in adipose tissue. It was also shown that fat pad biopsy material can be used to analyze acetylation status of histone proteins. This finding taken together with our results describing prediction and assessment of tumor response to targeted agents strongly suggest that sequential fat pad biopsy can be useful at showing inhibition of target pathway inhibition in the fat and vascular endothelial cells in vivo. The fat pad biopsies, by its ease of access, will enable to optimize pharmacodynamic methods to detect inhibition of the expressed targets and their corresponding pathways in vivo in a quantitative manner. Fat pad studies may also provide broad indications of the appropriate dose range and the best scheduling in individual patients. Combination of fat pad analysis with assessment of tumor pharmacodynamic end points would enable us to determine the pharmacokinetic and pharmacodynamic effects of targeted therapeutics and HDAC inhibitors in patients as a first step towards the personalized medicine.

Obesity and type 2 diabetes are the most prevalent and serious metabolic diseases; they affect more than 50% of adults in the USA. These conditions are associated with a chronic inflammatory response characterized by abnormal cytokine production, increased acute-phase reactants and other stress-induced molecules. Many of these alterations are initiated and to reside within adipose tissue. Elevated production of tumor necrosis factor by adipose tissue decreases sensitivity to insulin. Several lines of evidence suggest that dysregulation of signaling pathways involving JNK, PI3K/AKT/GSK3, MEK/ERK are causally linked to aberrant metabolic control in obesity and insulin resistance in type 2 diabetes.

In vivo and ex vivo monitoring of tissue response obtained by fat pad biopsy can also be potentially used to assess the effect of hormones, such as insulin, and other cytokines in metabolic diseases such as obesity and type 2 diabetes to determine patients' sensitivity and resistance to therapeutic and preventive applications.

Taking a series of repeat biopsies or fine needle aspirates of a tumor and adipose tissue during the course of therapy can provide information about treatment-induced changes in expression and activation of signaling and metabolic proteins and help monitor patient response to therapy. It is expected that this approach will also further our understanding of the molecular mechanisms that determine a patient's response or resistance to therapy in metabolic and neoplastic diseases, may facilitate investigation of molecular biology of disease response, and may provide useful information towards the development of new therapeutic and preventive agents.

### INCORPORATION BY REFERENCE

The contents of all references, patents, pending patent applications and published patents, cited throughout this application are hereby expressly incorporated by reference.

### LITERATURE CITED

The following documents have been cited above by reference to indicated sequential numbers or otherwise.
1. Arteaga, C. L. and Baselga, J. Tyrosine kinase inhibitors: why does the current process of clinical development not apply to them? Cancer Cell, 5: 525-531, 2004.
2. Baselga, J. and Arribas, J. Treating cancer's kinase ‘addiction’. Nat Med, 10: 786-787, 2004.
3. Sawyers, C. Targeted cancer therapy. Nature, 432: 294-297, 2004.
4. Kantarjian, H., Sawyers, C., Hochhaus, A., Guilhot, F., Schiffer, C., Gambacorti-Passerini, C., Niederwieser, D., Resta, D., Capdeville, R., Zoellner, U., Talpaz, M., Druker, B., Goldman, J., O'Brien, S. G., Russell, N., Fischer, T., Ottmann, 0., Cony-Makhoul, P., Facon, T., Stone, R., Miller, C., Tallman, M., Brown, R., Schuster, M., Loughran, T., Gratwohl, A., Mandelli, F., Saglio, G., Lazzarino, M., Russo, D., Baccarani, M., and Mona, E. Hematologic and cytogenetic responses to imatinib mesylate in chronic myelogenous leukemia. N Engl J Med, 346: 645-652, 2002.
5. O'Brien, S. G., Guilhot, F., Larson, R. A., Gathmann, I., Baccarani, M., Cervantes, F., Cornelissen, J. J., Fischer, T., Hochhaus, A., Hughes, T., Lechner, K., Nielsen, J. L., Rousselot, P., Reiffers, J., Saglio, G., Shepherd, J., Simonsson, B., Gratwohl, A., Goldman, J. M., Kantarjian, H., Taylor, K., Verhoef, G., Bolton, A. E., Capdeville, R., and Druker, B. J. Imatinib compared with interferon and low-dose cytarabine for newly diagnosed chronic-phase chronic myeloid leukemia. N Engl J Med, 348: 994-1004, 2003.
6. Bell, D. W., Lynch, T. J., Haserlat, S. M., Harris, P. L., Okimoto, R. A., Brannigan, B. W., Sgroi, D. C., Muir, B., Riemenschneider, M. J., lacona, R. B., Krebs, A. D., Johnson, D. H., Giaccone, G., Herbst, R. S., Manegold, C., Fukuoka, M., Kris, M. G., Baselga, J., Ochs, J. S., and Haber, D. A. Epidermal growth factor receptor mutations and gene amplification in non-small-cell lung cancer: molecular analysis of the IDEAL/INTACT gefitinib trials. J Clin Oncol, 23: 8081-8092, 2005.
7. Hirsch, F. R., Varella-Garcia, M., McCoy, J., West, H., Xavier, A. C., Gumerlock, P., Bunn, P. A., Jr., Franklin, W. A., Crowley, J., and Gandara, D. R. Increased epidermal growth factor receptor gene copy number detected by fluorescence in situ hybridization associates with increased sensitivity to gefitinib in patients with bronchioloalveolar carcinoma subtypes: a Southwest Oncology Group Study. J Clin Oncol, 23: 6838-6845, 2005.
8. Cappuzzo, F., Hirsch, F. R., Rossi, E., Bartolini, S., Ceresoli, G. L., Bemis, L., Haney, J., Witta, S., Danenberg, K., Domenichini, I., Ludovini, V., Magrini, E., Gregorc, V., Doglioni, C., Sidoni, A., Tonato, M., Franklin, W. A., Crino, L., Bunn, P. A., Jr., and Varella-Garcia, M. Epidermal growth factor receptor gene and protein and gefitinib sensitivity in non-small-cell lung cancer. J Natl Cancer Inst, 97: 643-655, 2005.
9. Pao, W., Wang, T. Y., Riely, G. J., Miller, V. A., Pan, Q., Ladanyi, M., Zakowski, M. F., Heelan, R. T., Kris, M. G., and Varmus, H. E. KRAS mutations and primary resistance of lung adenocarcinomas to gefitinib or erlotinib. PLoS Med, 2: e17, 2005.
10. Mellinghoff, I. K., Wang, M. Y., Vivanco, I., Haas-Kogan, D. A., Zhu, S., Dia, E. Q., Lu, K. V., Yoshimoto, K., Huang, J. H., Chute, D. J., Riggs, B. L., Horvath, S., Liau, L M., Cavenee, W. K., Rao, P. N., Beroukhim, R., Peck, T. C., Lee, J. C., Sellers, W. R., Stokoe, D., Prados, M., Cloughesy, T. F., Sawyers, C. L., and Mischel, P. S. Molecular determinants of the response of glioblastomas to EGFR kinase inhibitors. N Engl J Med, 353: 2012-2024, 2005.
11. Ogino, S., Meyerhardt, J. A., Cantor, M., Brahmandam, M., Clark, J. W., Namgyal, C., Kawasaki, T., Kinsella, K., Michelini, A. L., Enzinger, P. C., Kulke, M. H., Ryan, D. P., Loda, M., and Fuchs, C. S. Molecular alterations in tumors and response to combination chemotherapy with gefitinib for advanced colorectal cancer. Clin Cancer Res, 11: 6650-6656, 2005.
12. Haas-Kogan, D. A., Prados, M. D., Tihan, T., Eberhard, D. A., Jelluma, N., Arvold, N. D., Baumber, R., Lamborn, K. R., Kapadia, A., Malec, M., Berger, M. S., and Stokoe, D. Epidermal growth factor receptor, protein kinase B/Akt, and glioma response to erlotinib. J Natl Cancer Inst, 97: 880-887, 2005.
13. Heinrich, M. C., Corless, C. L., Demetri, G. D., Blanke, C. D., von Mehren, M., Joensuu. H., McGreevey, L. S., Chen, C. J., Van den Abbeele, A. D., Druker, B. J., Kiese, B., Eisenberg, B., Roberts, P. J., Singer, S., Fletcher, C. D., Silberman, S., Dimitrijevic, S., and Fletcher, J. A. Kinase mutations and imatinib response in patients with metastatic gastrointestinal stromal tumor. J Clin Oncol, 21: 4342-4349, 2003.
14. Paez, J. G., Janne, P. A., Lee, J. C., Tracy, S., Greulich, H., Gabriel, S., Herman, P., Kaye, F. J., Lindeman, N., Boggon, T. J., Naoki, K., Sasaki, H., Fujii, Y., Eck, M. J., Sellers, W. R., Johnson, B. E, and Meyerson, M. EGFR mutations in lung cancer: correlation with clinical response to gefitinib therapy. Science, 304: 1497-1500, 2004.
15. Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., Louis, D. N., Christiani, D. C., Settleman, J., and Haber, D. A. Activating mutations in the epidermal growth factor receptor underlying responsiveness of non-small-cell lung cancer to gefitinib. N Engl J Med, 350:2129-2139, 2004.
16. Twombley, R. Identity crisis: finding, defining and integrating biomarkers still a challenge. JNCI, 98:11-12, 2006.
17. Jimeno, A., Rubio-Viqueira, B., Amador, M. L, Oppenheimer, D., Bouraoud, N., Kulesza, P., Sebastiani, V., Maitra, A., and Hidalgo, M. Epidermal growth factor receptor dynamics influences response to epidermal growth factor receptor targeted agents. Cancer Res, 65: 3003-3010, 2005.
18. deGraffenried, L. A., Friedrichs, W. E., Russell, D. H., Donzis, E. J., Middleton, A. K., Silva, J. M., Roth, R. A., and Hidalgo, M. Inhibition of mTOR activity restores tamoxifen response in breast cancer cells with aberrant Akt Activity. Clin Cancer Res, 10: 8059-8067, 2004.
19. Grunwald, V., DeGraffenried, L., Russel, D., Friedrichs, W. E., Ray, R. B., and Hidalgo, M. Inhibitors of mTOR reverse doxorubicin resistance conferred by PTEN status in prostate cancer cells. Cancer Res, 62: 6141-6145, 2002.
20. Adjei, A. A. and Hidalgo, M. Intracellular signal transduction pathway proteins as targets for cancer therapy. J Clin Oncol, 23:5386-5403, 2005.
21. Magne, N., Fischel, J. L., Dubreuil, A., Formento, P., Poupon, M. F., Laurent-Puig, P., and Milano, G. Influence of epidermal growth factor receptor (EGFR), p53 and intrinsic MAP kinase pathway status of tumour cells on the antiproliferative effect of ZD1839 (“Iressa”). Br J Cancer, 86: 1518-1523, 2002.
22. Agbunag, C. and Bar-Sagi, D. Oncogenic K-ras drives cell cycle progression and phenotypic conversion of primary pancreatic duct epithelial cells. Cancer Res, 64: 5659-5663, 2004.
23. Hamburger, A. W. and Salmon, S. E. Primary bioassay of human tumor stem cells. Science, 197:461-463, 1977.
24. Gerhardt, R. T., Perras, J. P., Sevin, B. U., Petru, E., Ramos, R., Guerra, L, and Averette, H. E. Characterization of in vitro chemosensitivity of perioperative human ovarian malignancies by adenosine triphosphate chemosensitivity assay. Am J Obstet Gynecol, 165: 245-255, 1991.
25. Kern, D. H. and Weisenthal, L. M. Highly specific prediction of antineoplastic drug resistance with an in vitro assay using suprapharmacologic drug exposures. J Natl Cancer Inst, 82: 582-588, 1990.
26. Meitner, P. A. The fluorescent cytoprint assay: a new approach to in vitro chemosensitivity testing. Oncology (Huntingt), 5: 75-81; discussion 81-72, 85, 88, 1991.
27. Andreotti, P. E., Cree, I. A., Kurbacher, C. M., Hartmann, D. M., Linder, D., Harel, G., Gleiberman, I., Caruso, P. A., Ricks, S. H., Untch, M., and et al. Chemosensitivity testing of human tumors using a microplate adenosine triphosphate luminescence assay: clinical correlation for cisplatin resistance of ovarian carcinoma. Cancer Res, 55: 5276-5282, 1995.
28. Hirano, Y., Ushiyama, T., Suzuki, K., and Fujita, K. Clinical application of an in vitro chemosensitivity test, the Histoculture Drug Response Assay, to urological cancers: wide distribution of inhibition rates in bladder cancer and renal cell cancer. Urol Res, 27: 483-488, 1999.
29. Sharma, S., Neale, M. H., Di Nicolantonio, F., Knight, L. A., Whitehouse, P. A., Mercer, S. J., Higgins, B. R., Lamont, A., Osborne, R., Hindley, A. C., Kurbacher, C. M., and Cree, I. A. Outcome of ATP-based tumor chemosensitivity assay directed chemotherapy in heavily pre-treated recurrent ovarian carcinoma. BMC Cancer, 3:19, 2003.
30. Campiglio, M., Locatelli, A., Olgiati, C., Normanno, N., Somenzi, G., Vigano, L., Fumagalli, M., Menard, S., and Gianni, L. Inhibition of proliferation and induction of apoptosis in breast cancer cells by the epidermal growth factor receptor (EGFR) tyrosine kinase inhibitor ZD1839 (‘Iressa’) is independent of EGFR expression level. J Cell Physiol, 198: 259-268, 2004.
31. Bishop, P. C., Myers, T., Robey, R., Fry, D. W., Liu, E. T., Blagosklonny, M. V., and Bates, S. E. Differential sensitivity of cancer cells to inhibitors of the epidermal growth factor receptor family. Oncogene, 21: 119-127, 2002.
32. Scartozzi M, B. I., Berardi R, Mandolosi A, Fabris G, Cascinu S Epidermal growth factor receptor (EGFR) status in primary colorectal tumors does not correlate with EGFR expression in related metastatic sites: implications for treatment with EGFR-targeted monoclonal antibodies. J Clin Oncol, 22: 4772-4778, 2004.
33. De Pas T, P. G., de Braud F, Veronesi G, Cirigliano G, Leon M E Modulation of epidermal growth factor receptor (EGFR) status by chemotherapy in patients with locally advanced non small cell is rare. J Clin Oncol, 22: 4966-4970, 2004.
34. Cappuzzo, F., Magrini, E., Ceresoli, G. L., Bartolini, S., Rossi, E., Ludovini, V., Gregorc, V., Ligorio, C., Cancellieri, A., Damiani, S., Spreafico, A., Paties, C. T., Lombardo, L., Calandri, C., Bellezza, G., Tonato, M., and Crino, L. Akt phosphorylation and gefitinib efficacy in patients with advanced non-small-cell lung cancer. J Natl Cancer Inst, 96: 1133-1141, 2004.
35. Han, S. W., Kim, T. Y., Hwang, P. G., Jeong, S., Kim, J., Choi, I. S., Oh, D. Y., Kim, J. H., Kim, D. W., Chung, D. H., Im, S. A., Kim, Y. T., Lee, J. S., Heo, D. S., Bang, Y. J., and Kim, N. K. Predictive and prognostic impact of epidermal growth factor receptor mutation in non-small-cell lung cancer patients treated with gefitinib. J Clin Oncol, 23: 2493-2501, 2005.
36. Pao, W., Miller, V. A., Venkatraman, E., and Kris, M. G. Predicting sensitivity of non-small-cell lung cancer to gefitinib: is there a role for P-Akt? J Natl Cancer Inst, 96: 1117-1119, 2004.
37. Peralba, J. M., DeGraffenried, L., Friedrichs, W., Fulcher, L., Grunwald, V., Weiss, G., and Hidalgo, M. Pharmacodynamic Evaluation of CCI-779, an Inhibitor of mTOR, in Cancer Patients. Clin Cancer Res, 9: 2887-2892, 2003.
38. Baselga, J., Albanell, J., Ruiz, A., Lluch, A., Gascon, P., Guillem, V., Gonzalez, S., Sauleda, S., Marimon, I., Tabemero, J. M., Koehler, M. T., and Rojo, F. Phase II and Tumor Pharmacodynamic Study of Gefitinib in Patients with Advanced Breast Cancer. J Clin Oncol, 2005.
39. Schrag, D., Garewal, H. S., Burstein, H. J., Samson, D. J., Von Hoff, D. D., and Somerfield, M. R. American Society of Clinical Oncology Technology Assessment: chemotherapy sensitivity and resistance assays. J Clin Oncol, 22: 3631-3638, 2004.
40. Pelosi, G., Bresaola, E., Rodella, S., Manfrin, E., Piubello, Q., Schiavon, I., and Iannucci, A. Expression of proliferating cell nuclear antigen, Ki-67 antigen, estrogen receptor protein, and tumor suppressor p53 gene in cytologic samples of breast cancer: an immunochemical study with clinical, pathobiological, and histologic correlations. Diagn Cytopathol, 11: 131-140, 1994.
41. Makris, A., Allred, D. C., Powles, T. J., Dowsett, M., Fernando, I. N., Trott, P. A., Ashley, S. E., Ormerod, M. G., Titley, J. C., and Osborne, C. K. Cytological evaluation of biological prognostic markers from primary breast carcinomas. Breast Cancer Res Treat, 44: 65-74, 1997.
42. Rao, J. Y., Apple, S. K., Hemstreet, G. P., Jin, Y., and Nieberg, R. K. Single cell multiple biomarker analysis in archival breast fine-needle aspiration specimens: quantitative fluorescence image analysis of DNA content, p53, and G-actin as breast cancer biomarkers. Cancer Epidemiol Biomarkers Prey, 7: 1027-1033, 1998.
43. Nizzoli, R., Bozzetti, C., Naldi, N., Guazzi, A., Gabrielli, M., Michiara, M., Camisa, R., Barilli, A., and Cocconi, G. Comparison of the results of immunocytochemical assays for biologic variables on preoperative fine-needle aspirates and on surgical specimens of primary breast carcinomas. Cancer, 90: 61-66, 2000.
44. Assersohn, L., Gangi, L., Zhao, Y., Dowsett, M., Simon, R., Powles, T. J., and Liu, E. T. The feasibility of using fine needle aspiration from primary breast cancers for cDNA microarray analyses. Clin Cancer Res, 8: 794-801, 2002.
45. Kuner, R., Pollow, K., Lehnert, A., Pollow, B., Scheler, P., Krummenauer, F., Casper, F., and Hoffmann, G. [Needle biopsy vs. conventional surgical biopsy-biochemical analysis of various prognostic factors]. Zentralbl Gynakol, 122: 160-164, 2000.
46. Pusztai, L., Ayers, M., Stec, J., Clark, E., Hess, K., Stivers, D., Damokosh, A., Sneige, N., Buchholz, T. A., Esteva, F. J., Arun, B., Cristofanilli, M., Booser, D., Rosales, M., Valero, V., Adams, C., Hortobagyi, G. N., and Symmans, W. F. Gene expression profiles obtained from fine-needle aspirations of breast cancer reliably identify routine prognostic markers and reveal large-scale molecular differences between estrogen-negative and estrogen-positive tumors. Clin Cancer Res, 9: 2406-2415, 2003.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for assessing the therapeutic potential of one or more chemotherapeutic or metabolic agents, the method comprising:
isolating a fine needle aspiration biopsy from living tissue;
preparing the fine needle aspiration biopsy for assessment;
treating at least a portion of the fine needle aspiration biopsy;
determining an extent of protein expression and post-translational modifications of a plurality of metabolic proteins in the at least a portion of the fine needle aspiration biopsy; and
comparing the extent of protein expression and post-translational modifications of the plurality of metabolic proteins in the at least a portion of the fine needle aspiration biopsy treated during the step of treating with a reference, untreated sample.

2. The method of claim 1, wherein the step of preparing includes smearing at least a portion of the fine needle aspiration biopsy on a surface; and
air-drying the at least a portion of the fine needle aspiration biopsy smeared on the surface in the step of preparing prior to the step of treating.

3. The method of claim 1, wherein the steps are completed sequentially in the following order isolating, preparing, treating, determining and comparing.

4. The method of claim 1, further comprising selecting the plurality of metabolic proteins such that the step of comparing assesses the therapeutic potential of one or more metabolic agents for treating type II diabetes.

5. The method of claim 1, further comprising selecting the plurality of metabolic proteins such that the step of comparing assesses the therapeutic potential of one or more chemotherapeutic agents for treating cancer.

6. The method of claim 5, wherein the step of selecting includes selecting the plurality of metabolic proteins such that phosphorylation of one or more of the plurality of metabolic proteins indicates whether the one or more chemotherapeutic agents are not going to be effective in treating cancer in the patient.

7. The method of claim 5, wherein the step of selecting includes selecting the plurality of metabolic proteins such that tumor resistance to one or more of the one or more chemotherapeutic agents is determined.

8. The method of claim 7, wherein the steps of isolating, preparing, treating, determining and comparing are repeated during a course of treatment to monitor the effect of the chemotherapeutic agents in vivo.

9. The method of claim 1, wherein the plurality of proteins are selected to include an extracellular signal-regulated kinase inhibitor.

10. The method of claim 9, wherein the plurality of proteins are selected to include a mitogen-activated protein kinase kinase (MEK).

11. The method of claim 9, wherein the plurality of proteins are selected to include S6 ribosomal protein.

12. The method of claim 9, wherein the plurality of proteins are selected to include a protein kinase B (AKT).

13. The method of claim 1, wherein the step of treating is conducted ex vivo.

14. The method of claim 13, further comprising a step of staining, prior to the steps of determining and comparing.

15. The method of claim 14, further comprising a step of sequencing genomic tissue after the step of staining.

16. The method of claim 1, wherein the living tissue is fat pad tissue.

17. The method of claim 16, wherein the plurality of metabolic proteins includes histone proteins.

18. The method of claim 17, wherein the step of determining the post-translational modifications includes determining the acetylation status of the histone proteins.

19. The method of claim 1, wherein the plurality of metabolic proteins are selected to include a Jun N-terminal kinase (JNK), a protein kinase B (AKT), an extracellular signal-regulated kinase inhibitor (ERK), or a combination thereof.
